(19) United States
(12) Patent Application Publication
(10) Pub. No.: US 2026/0294934 A1
(43) Pub. Date: Oct. 1, 2026

(54) CORTICOSTEROID NANOSUSPENSIONS AND USES THEREOF

(71) Applicant: AIMMAX THERAPEUTICS, INC., Durham, NC (US)

(72) Inventors: DEREK J. NUNEZ, Durham, NC (US); LAURENE WANG, Durham, NC (US); JASON B. COOK, Durham, NC (US); Wei-Cheng Wayne LIAW, San Diego, CA (US)

(73) Assignee: AIMMAX THERAPEUTICS, INC., Durham, NC (US)

(21) Appl. No.: 19/479,787

(22) PCT Filed: Apr. 30, 2024
(86) PCT No.: PCT/US2024/026982

§ 371 Date: Oct. 29, 2025

Related U.S. Application Data
(60) Provisional application No. 63/463,113, filed on May 1, 2023.

(51) Int. Cl. A61K 31/573; A61K 9/00; A61K 9/12; A61K 47/02; A61K 47/12; A61K 47/18; A61K 47/26; A61K 47/34; A61K 47/38; A61K 47/46; A61P 17/02; A61P 19/02; A61P 27/16

(52) U.S. Cl. CPC A61K 31/573; A61K 9/0021; A61K 9/12; A61K 47/02; A61K 47/12; A61K 47/186; A61K 47/26; A61K 47/34; A61K 47/38; A61K 47/46; A61P 17/02; A61P 19/02; A61P 27/16

(57) ABSTRACT

Suspension formulations of nanoparticles of clobetasol propionate are described. The suspensions can be used therapeutically to treat skin and ocular burns; to enhance wound healing; to prevent or reduce hypertrophic scarring/keloids; to treat allergic rhinitis/sinusitis, asthma, inner ear disorders including hearing loss, tinnitus, or vertigo, tenosynovitis, tendinitis, enthesitis or arthritis.

### FIELD OF THE INVENTION

The invention described herein relates to suspension formulations of nanoparticles of corticosteroids, such as clobetasol propionate, and methods for treating dermal or ocular burn injuries, abnormal wound healing, allergic rhinitis/sinusitis, asthma, inner ear disorders, tenosynovitis, tendinitis, enthesitis and arthritis.

### BACKGROUND

### Treatment of Burn Injuries

A burn is an injury to the skin or other tissue, including tissues in the eye, from many causes that damages cellular structures. Burn injuries can vary in severity depending on the extent of tissue damage, sometimes leading to extensive tissue inflammation, cell death and necrosis. Dermal burn treatments depend on the type of burn and their severity. In more severe cases intensive treatments are required to improve the healing process and reduce complications such as hypertrophic scarring, skin contractures, keloids, and deformities.

Ocular burns can be especially problematic because injuries can cause irreversible damage to the cornea and other structures in the eye, greatly affecting vision. The cornea is the major refractive tissue at the front of the eye. It is normally avascular and is composed of a thin anterior epithelial layer, a thin posterior endothelial layer and a highly complex stromal layer that makes up approximately 90% of the corneal thickness. Transparency of the cornea is essential to allow light to enter the eye and reach the light-sensitive cells in the retina at the back of the eyeball. Corneal transparency is achieved by having an acellular stroma with a highly ordered structural organization of several types of collagen fibers and other extracellular matrix molecules. When this complex stromal organization is disrupted by mechanical damage, inflammation, edema or neovascularization, the cornea becomes increasingly cloudy and vision can be markedly impaired. Thus, to preserve vision it is necessary to decrease and/or prevent inflammation, edema, neovascularization and disorganization of stromal matrix proteins after a burn injury. Current treatments are not completely effective at restoring those structures to normal.

Regardless of the type of burn, the healing progress should be closely monitored because inflammation and abnormal repair processes can lead to long-term complications. For example, in the case of skin burn injuries, hypertrophic scaring, skin contractures, and abnormal pigmentation can have a significant impact on the quality of life of a patient. Ocular burn injuries can affect the conjunctiva, sclera, cornea, and even extraocular tissues such as the eyelids and can be an ophthalmologic emergency requiring rapid assessment and intervention to preserve vision by minimizing inflammation and the accumulation of materials that impede the entry of light into the eye. There exists a continuing need for treatments and treatment methods for burn injuries, including methods focused on reducing the extent of scarring, the speed of the healing process and the restoration and preservation of vision.

### Treatment of Abnormal Wound Healing

Skin wounds typically heal in four phases; hemostasis followed by inflammation, cell proliferation and then remodeling. Scar tissue forms during the remodeling phase. Normally, a small superficial wound heals by forming new skin that blends with the surrounding tissue. Deeper wounds into the dermis layer and below require more extensive connective tissue remodeling and collagen deposition to repair the wound and this can become a permanent scar. Some types of skin injuries result in prolonged and excessive inflammation and are associated with abnormal wound healing. There is also a genetic predisposition to excessive scarring and keloid formation. Current treatments for hypertrophic and keloid scars, including laser therapy, antiproliferative drug injections, such as bleomycin and 5-fluorouracil, and cryotherapy, provide only limited efficacy. Injection of conventional steroid formulations are limited because the suboptimal efficacy necessitates repeated painful injections but these do not prevent the progression of the scarring, disfigurement and impaired mobility. Therefore, there is a need for more effective treatments to prevent disfiguring scarring.

### Treatment of Allergic Rhinitis/Sinusitis

Allergic rhinitis and sinusitis (rhinitis/sinusitis) are common types of allergies that can be caused by seasonal or nonseasonal allergens. Acute hypersensitivity reaction to an allergen is characterized by sneezing, runny nose, itchy eyes, nose and palate, nasal and sinus congestion, post-nasal drip, cough, airway constriction and other debilitating symptoms. Though conventional treatments may manage low-grade symptoms of allergic rhinitis/sinusitis, in some cases, the treatments are insufficient, and intervention is required to prevent severe complications such as airway constriction. There is a continuing need for therapeutics and treatment methods for allergic rhinitis/sinusitis.

### Treatment of Asthma

Asthma is a chronic disease affecting the lungs that is characterized by the presence of inflammation, narrowing of the airways and bronchoconstriction which cause wheezing, chest tightness, shortness of breath, and coughing. The causes of asthma are not completely understood, but there may be a genetic predisposition to environmental triggers such as allergens, tobacco smoke or other noxious gases and airborne particulate matter. The release of inflammatory mediators post exposure to these factors contribute to bronchospasm, airway edema, and abnormal mucous production resulting in the characteristic asthma symptoms. In some cases, conventional steroid formulations can be used, but generally must be co-administered with beta adrenergic receptor agonists to reduce airway inflammation and asthma symptoms. However, in many cases, the current medications are insufficient to prevent an asthma attack, thus requiring excessively high doses and the coadministration of long-acting beta agonists. Thus, there is a continuing need for therapeutics and treatment methods for asthma.

### Inner Ear Disorders

Inner ear disorders can present with hearing loss, vertigo and tinnitus that affects many adults, especially the elderly. When tinnitus, hearing loss and vertigo occur together they are referred to as Menière's disease. These inner ear disorders are frequently accompanied by endolymphatic hydrops which is an expansion of the endolymph fluid compartment of the inner ear. A pharmacological treatment has not yet been approved by the US Food and Drug Administration to prevent or treat hearing loss, vertigo or tinnitus. Because the etiology of these symptoms is unclear, many of the therapeutic treatments that have been proposed are ineffective or have limitations or severe side effects. Thus, there is a continuing need for treatments and treatment methods for hearing loss, tinnitus, and vertigo.

### Treatment of Tenosynovitis, Tendinitis, Enthesitis and Arthritis

Tenosynovitis is an inflammation of tendons within the synovial sheath, while enthesitis is inflammation of a tendon or ligament where it attaches to bone. Tendons moving inside their sheaths or at their attachments to bone can be irritated by trauma or repetitive or excessive movements causing inflammation which is painful and can severely limit function. Chronic inflammation can produce swelling and or fibrosis in the tendon sheath reducing the space for the tendons, causing for example “trigger finger” or nerve compression as in the case of the carpal tunnel syndrome. The repetitive joint motion or the impact of vibrating tools can initiate the inflammatory process in the tendon sheath or attachment point and this can occur in the hands or feet affecting individuals in many types of professions.

Medical treatment of tenosynovitis, tendinitis and enthesitis includes the use of splints to reduce movement of the tendon and corticosteroid injections. In some cases, surgery is required when pain and impaired function of the tendon have not responded to rest and anti-inflammatory treatments, but it can be very painful and can produce additional fibrosis and long-term complications.

Arthritis is the most common cause of disability in the US, causing pain and inflammation in a joint that affects more than 50 million people of all ages, including children. Osteoarthritis and rheumatoid arthritis are the 2 most common types of arthritis, but there are other types such as ankylosing spondylitis, gout and psoriatic arthritis. Osteoarthritis is the most common type of arthritis, often developing in middle aged or older persons, though osteoarthritis can occur at any age after injury to a joint. Osteoarthritis initially affects the smooth cartilage lining of the joint, restricting movement and causing pain and stiffness. Continued damage of the cartilage lining leads to swelling of the joint and can lead to formation of bony osteophytes and disruption of joint function. Rheumatoid arthritis, less common than osteoarthritis, is an immune-mediated damage of the joint tissues which starts in the synovial lining of the joint and leads to pain, swelling and joint deformity.

Symptoms of arthritis include joint pain, tenderness and stiffness, inflammation in and around the joints, restricted movement of the joints, warm red skin over the affected joint and weakness and muscle wasting. There are no cures for arthritis, though current treatments can help slow down joint damage including lifestyle changes, medicines, physical therapy and surgery. The treatment options depend on the severity of the arthritis, the symptoms and the patient's overall health, but in many cases, the symptoms and signs are not improved. Surgery, including fusion of bones to immobilize a joint and reduce pain caused by movement, or artificial joint replacement to preserve joint function and movement, remains the only option in certain severe cases of arthritis. Therefore, there is a continuing need for treatments and treatment methods for osteoarthritis, rheumatoid arthritis, and other less common types of arthritis.

### SUMMARY OF THE INVENTION

In one illustrative embodiment of the invention, compositions comprising corticosteroid and/or glucocorticoid nanoparticle suspensions, and uses thereof for the manufacture of medicaments and compositions for treating diseases are described herein. Illustrative diseases include dermal or ocular burn injuries, abnormal wound healing, allergic rhinitis/sinusitis, asthma, inner ear disorders, tenosynovitis, tendinitis, enthesitis and arthritis.

It has been discovered herein that dermal or ocular burn injuries, abnormal wound healing, allergic rhinitis/sinusitis, asthma, inner ear disorders, including hearing loss, tinnitus and vertigo, tenosynovitis, tendinitis, enthesitis and arthritis, including osteoarthritis and rheumatoid arthritis, can be more effectively treated with nanosuspension formulations of glucocorticoids, such as but not limited to clobetasol propionate, and the like. Such glucocorticoids are formulated as a nanoparticle suspension that may be applied topically, administered orally, injected parenterally, injected intra-tympanically; injected into a joint, synovial space, or around a tendon insertion; or administered as a spray, aerosol, or drops into the nasal cavity, respiratory system or external ear canal.

In another embodiment, compositions comprising nanosuspensions of glucocorticoids are described herein for use in the treatment of dermal or ocular burn injuries, abnormal wound healing, allergic rhinitis/sinusitis, asthma, inner ear disorders, including hearing loss, vertigo and tinnitus, tenosynovitis, tendinitis, enthesitis and arthritis.

In another embodiment, methods for treating dermal or ocular burn injuries, abnormal wound healing, allergic rhinitis/sinusitis, asthma, inner ear disorders s are described herein. The methods include administering an effective amount of a nanosuspension of a glucocorticoid to a subject in need.

In another embodiment, uses of compositions comprising nanosuspensions of glucocorticoids are described herein in the manufacture of medicaments for treating dermal or ocular burn injuries, abnormal wound healing, allergic rhinitis/sinusitis, asthma, inner ear disorders, tenosynovitis, tendinitis, enthesitis and arthritis.

In another embodiment, packaged articles and kits that include compositions comprising nanosuspensions of glucocorticoids are described herein. The packaged articles and kits include instructions for using the nanosuspensions in the treatment of dermal or ocular burn injuries, abnormal wound healing, allergic rhinitis/sinusitis, asthma, inner ear disorders, tenosynovitis, tendinitis, enthesitis and arthritis.

Corticosteroids have been used clinically to treat hearing loss, tinnitus and vertigo, but with only limited success. Glucocorticoid receptors are expressed in the inner ear, including hair cells and spiral ganglion neurons. Glucocorticoids are thought to preserve hearing and treat inner ear disease via multiple mechanisms of action including reduction of inner ear inflammation and reactive oxidative species, activation of cell survival pathways, and reduction of hair cell and spiral ganglion neuron apoptosis. However, the current corticosteroid treatment paradigms are suboptimal because of limited efficacy that may be related to a combination of factors, including inadequate potency of the corticosteroid and insufficient concentrations of the corticosteroid in the inner ear due to poor delivery or limited penetration. As described herein, embodiments of this invention include the use of a nanoparticle formulation containing clobetasol propionate, the most potent glucocorticoid used in clinical practice, to increase the penetration and concentration of this glucocorticoid in the inner ear tissues and fluids.

Embodiments of the invention include drops, sprays, mists, or aerosols for treating various types of tissue burns, such as burns due to heat, a chemical exposure, sun exposure, or mechanical damage. The drops, sprays, mists, and aerosols are optionally included with or in an applicator, a burn dressing, and the like. In other embodiments, the drops, sprays, mists, and aerosols minimize or alleviate pain, improve healing, reduce scarring, and/or improve vision.

Embodiments of the invention include injections into a hypertrophic scar or keloid to reduce connective tissue accumulation, deformation of the skin, contractures and improve cosmetic appearance. Embodiments of the invention include a spray, mist, aerosol or drops into the nasal cavity to treat allergic rhinitis/sinusitis or inhaled into the lungs to treat asthma. In other embodiments, the spray, mist, aerosol or drops reduce the symptoms of allergic rhinitis/sinusitis or prevent, reduce or stop asthma attacks.

Embodiments of the invention include a spray or drops applied onto the tympanic membrane through the external ear canal to treat inner ear disorders, specifically hearing loss, tinnitus and vertigo. Embodiments of the invention include intra-tympanic injection or intranasal administration with a spray, aerosol or drops to treat inner ear disorders, specifically hearing loss, tinnitus and vertigo. Without being bound by theory, it is believed herein that treatment failures with current medications arise due to insufficient potency and limited penetration into inner ear tissues and fluids and the nanoparticle suspension o highly potent glucocorticoids, such as clobetasol propionate, described herein, achieve greater penetration into the inner ear.

Embodiments of the invention include a sterile injection into the synovial space to treat tenosynovitis, tendinitis or into or around a tendon/ligament insertion to treat enthesitis. Embodiments of the invention include a sterile injection into a joint to treat arthritis.

In another illustrative embodiment, suspension formulations in the form of sprays, mists, aerosols, injectables, and drops containing about 0.025% to about 0.15% w/v clobetasol propionate nanoparticles are described herein. The suspension formulations can include additional agents, including but not limited to preservatives, antiseptics and/or disinfectants, and the like.

In another embodiment, processes for manufacturing nanoparticle suspensions of corticosteroids and/or glucocorticoids, such as clobetasol propionate are described herein.

It is to be understood that the compositions, kits, and methods described herein may include clobetasol propionate, or one or more alternative and/or additional corticosteroids and/or glucocorticoids, including but not limited to dexamethasone, difluprednate, triamcinolone, betamethasone dipropionate, prednisolone, and the like.

Other features and advantages of invention described herein will become apparent from the detailed description, taken in conjunction with the accompanying drawings, which further illustrate the invention by way of non-limiting examples.

### BRIEF DESCRIPTION OF THE DRAWINGS

FIG. 1 shows a flowchart of steps employed to produce illustrative suspension formulations of nanoparticles of clobetasol propionate.

FIGS. 2A-2D show the efficacy of CPN 0.1% following ocular alkali burn in New Zealand White rabbits. A statistical difference compared to vehicle is indicated by *(p<0.05).

FIGS. 3A-3C show the efficacy of CPN 0.1% in treating skin burn wounds in Yorkshire-cross pigs. A statistical difference compared to untreated control is indicated by *(p<0.05).

FIGS. 4A-4D show the efficacy of CPN 0.1% in a rabbit ear hypertrophic scar model. A statistical difference compared to untreated control is indicated by *(p<0.05).

FIGS. 5A-5B show the efficacy of CPN 0.1% in a collagen-induced arthritis (CIA) model in male Lewis rats. A statistical difference compared to vehicle is indicated by *(p<0.05).

### DETAILED DESCRIPTION

The following illustrative clauses set forth additional embodiments of the invention described herein:

1. A method for treating a subject with a dermal or ocular burn, abnormal wound healing, allergic rhinitis/sinusitis, asthma or an inner ear disorder, including but not limited to hearing loss, tinnitus or vertigo; the method comprising applying a suspension formulation of nanoparticles of clobetasol propionate to the dermal or ocular burn, skin wound, nasal or sinus mucous membranes (for allergic rhinitis/sinusitis or inner ear disorders), airways and tissues in the lungs, or tympanic membrane of the ear of the subject.

2. The method of clause 1, wherein the formulation is applied using a dropper bottle, a spray bottle or device, an aerosol delivery device, or an inhaler.

3. The method of clause 1 or 2, wherein the dermal or ocular burn is a sun burn, a radiation burn, a radioactivity burn, an electrical burn, a boiling liquid burn, a friction burn, a chemical burn, burns of high temperature combustion from fire, flame, explosives, ignition of fuel or lubricants, battle field weapons, or a combination of thereof; and optionally wherein the treatment of the ocular burn includes long-term improvement of vision and/or visual acuity following ocular burn injury.

4. The method of clause 1 or 2, wherein the abnormal wound healing is caused by a burn, inflammation, infection or trauma including surgical incision, and optionally wherein the treatment includes reducing and/or preventing hypertrophic scarring.

5. The method of clause 1 or 2, wherein the allergic rhinitis/sinusitis or asthma is caused by a chemical exposure, particle exposure or seasonal allergens; and inner ear disorder is caused by noise, trauma or an ototoxic chemical, and optionally wherein the treatment of allergic rhinitis/sinusitis or asthma includes the treatment or prevention of bronchospasm with reversible obstructive airway disease, the management of the nasal symptoms of perennial nonallergic rhinitis, the relief of the symptoms of seasonal and perennial allergic rhinitis, or any combination of the foregoing.

6. The method of any of the preceding clauses, wherein the suspension formulation of nanoparticles of clobetasol propionate reduces the complications of a dermal or ocular burn, abnormal wound healing, allergic rhinitis/sinusitis, asthma or an inner ear disorder, including, but not limited to, inflammation, pain, discomfort, swelling, corneal opacification, abnormal granulation and/or scarring, contractures, hypertrophic scarring, keloids, rhinorrhea, blocked nasal passages and sinuses, airway obstruction and respiratory distress, hearing loss, tinnitus and vertigo.

7. The method of any of the preceding clauses, wherein the suspension formulation of nanoparticles of clobetasol propionate improves wound healing, relieves allergic rhinitis/sinusitis or asthma, or reduces hearing loss, tinnitus or vertigo.

8. A method for treating a subject having a tendon/synovial disorder, including, but not limited to tendinitis, synovitis or enthesitis or arthritis; the method comprising introducing a sterile suspension formulation of nanoparticles of clobetasol propionate into the tympanic cavity, synovial space, tendon attachment or joint of the subject, and optionally wherein the treatment includes reducing inflammation around the joints, tendons and/or ligaments in the subject.

9. A method for treating a subject having an inner ear disorder, including, but not limited to hearing loss, tinnitus or vertigo; the method comprising introducing a sterile suspension formulation of nanoparticles of clobetasol propionate into the tympanic cavity, synovial space, tendon attachment or joint of the subject, and optionally wherein the treatment includes reducing cochlear hair cell loss, preserving hearing, or a combination of the foregoing.

10. The method of clause 8 or 9, wherein the formulation is introduced by injection, such as by using a syringe and needle.

11. The method of clause 8 or 9, wherein inner ear disorder is caused by noise, trauma or an ototoxic chemical

12. The method of clause 8 or 9, wherein the tendon/synovial disorder is caused by mechanical vibration, overuse, an autoimmune disorder, or a combination thereof, and/or is not caused, or not caused at least in part, by an infective agent or infection

13. The method of clause 8 or 9, wherein the arthritis is osteoarthritis, rheumatoid arthritis, ankylosing spondylitis, gout, or psoriatic arthritis, or a combination thereof and/or is not caused, or not caused at least in part, by an infective agent or infection.

14. The method of any of clauses 8 to 13, wherein the suspension formulation of nanoparticles of clobetasol propionate reduces the complications of an inner ear disorder, or a tendon/synovial disorder or arthritis, including, but not limited to, hearing loss, tinnitus, vertigo, inflammation, swelling, pain, discomfort, reduced mobility.

15. The method of any of clauses 8 to 14, wherein the suspension formulation of nanoparticles of clobetasol propionate reduces hearing, tinnitus, vertigo, or improves joint function or tendon function.

16. The method of any of the preceding clauses, wherein the suspension formulation of nanoparticles of clobetasol propionate is administered once or more times daily for up to 52 weeks; or at least once daily for 1 to 4 weeks.

17. A drop, spray, mist or aerosol or injection formulation comprising clobetasol propionate, wherein the clobetasol propionate is formulated as a suspension of nanoparticles.

18. The drop, spray, mist or aerosol or injection formulation of clause 17 for use in any of the methods of clauses 1 to 16.

19. The drop, spray, mist or aerosol or injection formulation of clause 17 or 18 and/or for use in any of the methods of clauses 1 to 16 further comprising sodium chloride, hydrogenated soybean lecithin, anhydrous citric acid, glycerin, Poloxamer 407, polyvinyl alcohol (PVA), boric acid, edetate disodium dihydrate, benzalkonium chloride, methylcellulose, or sodium citrate, such as trisodium citrate, or a combination thereof.

20. The drop, spray, mist or aerosol or injection formulation of any of clauses 17 to 19 and/or for use in any of the methods of clauses 1 to 16 further comprising an antioxidant, an antimicrobial agent, an anesthetic, an analgesic, a lubricant, a flavoring agent or a combination thereof.

21. A unit dose configured for administration by the transdermal, transmucosal, ocular or injection route; the unit dose comprising clobetasol propionate, wherein the clobetasol propionate is formulated as a sterile suspension of nanoparticles.

22. The unit dose of clause 21 for use in any of the methods of clauses 1 to 16.

23. The unit dose of clause 21 or 22 and/or for use in any of the methods of clauses 1 to 15 further comprising sodium chloride, hydrogenated soybean lecithin, anhydrous citric acid, glycerin, Poloxamer 407, polyvinyl alcohol (PVA), boric acid, edetate disodium dihydrate, benzalkonium chloride, methylcellulose, or sodium citrate, such as trisodium citrate, or a combination thereof.

### Definitions

The term “embodiment” generally refers to an illustrative example of the invention. The embodiment may describe one or more distinguishing features, structures, and/or characteristics, and the like, of the invention, and does not necessarily describe every feature, structure, and/or characteristic of the invention. The term “aspect” generally refers a particular feature, structure, or characteristic, and the like, of the invention described herein. It is to be understood that such aspects can appear alone or in combination; however, in each case such aspects may be optional and not necessarily included in the invention.

Unless otherwise defined, the terms used herein, including all technical and scientific terms, should be construed to generally have their ordinary meanings in the art, including when taken within the context of the disclosure, and in the specific context where each term is used.

The use of examples anywhere in this specification including examples of any terms discussed herein is illustrative only and is not intended to further limit the scope and meaning of the disclosure or of any exemplified term. Likewise, the disclosure is not limited to various embodiments given in this specification.

The terms “active agent,” “active ingredient,”. “active compound”, “active component”, “active pharmaceutical ingredient,” or “API” each generally refers to a substance, compound, chemical or molecule, which is biologically active or otherwise, induces a biological, physiological or pharmacological effect on a subject to which it is administered. In other words, “active agent” or “active ingredient” refers to a component or components of a composition to which the whole or part of the effect of the composition is attributed. A glucocorticoid can be a primary active agent, or in other words, the component of a composition to which the whole or part of the effect of the composition is attributed. An active agent can be a secondary agent, or in other words, the component of a composition to which an additional part and/or other effect of the composition is attributed.

The terms “effective amount” and “therapeutically effect amount” generally refer to the amount of the defined component sufficient to achieve the desired biological and/or therapeutic result. 10hat result can be the alleviation or amelioration of the signs, symptoms, or causes of a disease, or any other desired alteration of a biological system. When the desired result is a therapeutic response, it is to be understood that the effective amount will vary depending upon the specific disease or symptom to be treated or alleviated, the age, sex, body weight, general health and diet of the subject to be treated, the time of administration, the rate of excretion, possible drug combinations, the dosing regimen of the formulation, the size or location of the area to be treated and the form of administration, the severity of the disease condition, such as the type, degree and site of the burn wound, abnormal wound healing, allergic rhinitis/sinusitis, asthma, inner ear disorder, specifically hearing loss, tinnitus or vertigo, tenosynovitis, tendinitis, enthesitis or arthritis, the type and degree of the response to be achieved, whether other agents are used or not, the manner of administration and the like, all of which can be determined readily by one of ordinary skill in the art.

The terms “treating,” “treatment,” or “alleviation” each generally refer to both therapeutic treatment and prophylactic or preventative measures, wherein the object is to prevent or slow down (lessen) the targeted pathologic condition or disorder. A subject is successfully “treated” for a disease, disorder or condition if, after receiving a therapeutic amount of the nanoparticle suspension according to the methods described herein, the subject shows observable and/or measurable reduction in or absence of one or more signs and symptoms of a particular disease or condition. For example, for a burn injury, treatment or prevention may include a reduction in the size or severity of the burn wound, a reduction in pain, a quicker healing process and/or improved healing (e.g., a reduction in scaring/granulation/pigmentation). Other examples include: for abnormal wound healing, treatment or prevention may include a reduction in the size of the scar tissue, a quicker healing process and/or improved healing; for an ocular burn treatment or prevention may include reduction of corneal opacification and improved vision, for allergic rhinitis/sinusitis, treatment or prevention may include a reduction in nasal secretions and symptoms such as pruritus and nasal stuffiness; for asthma, treatment or prevention may include a reduction of bronchoconstriction, wheezing, shortness of breath, chest tightness, pain or coughing; for inner ear disorders, treatment or prevention may include an improvement of hearing, vertigo and tinnitus; for tenosynovitis, tendinitis, enthesitis and arthritis, treatment or prevention may include a reduction of pain, swelling and deformity and improved mobility.

The term “administration” generally refers to the introduction of an amount of a predetermined substance into or onto a subject by a certain suitable method. The compositions disclosed herein may be administered via any of the common routes, including topically, such as by application of a drop to the eye, spraying or by applying droplets onto the surface of the eye, skin or mucous membrane, or onto the tympanic membrane through the outer ear canal, or by injection into a synovial space, tendon or ligament insertion or joint or scar, or by intra-tympanic injection.

As used herein, the term “pharmaceutically acceptable” means that the composition is sufficient to achieve the therapeutic effects without deleterious side effects, and may be readily determined depending on the type, severity and site of the burn wound, abnormal wound healing, allergic rhinitis/sinusitis, asthma, inner ear disorder, specifically hearing loss, tinnitus or vertigo, tenosynovitis, tendinitis, enthesitis or arthritis, the subject's age, body weight, health conditions, gender, drug sensitivity, administration mode, administration frequency, duration of treatment, drugs used in combination or coincident with the composition disclosed herein, and other factors known in medicine.

The terms “burn” or “burn conditions” each generally refers to a wide range of maladies, including those resulting from, for example, excessive exposure to radiation, e.g., radioactivity and solar radiation resulting in sunburn, thermal radiation, welding flash, fires, electrical discharge, contact chemical exposure, friction, contact with hot objects such as cooking apparatus elements or hot fluids such as scalding water, steam vapor, hot oil, flame, etc., battlefield burn injuries, and the like. Such burns and burn conditions, though arising from varied sources, share common symptoms and disease progression.

The term “allergic rhinitis/sinusitis” generally refers to a range of such conditions, including those resulting from seasonal exposure to allergens such as pollens and those resulting from a chemical or particulate exposure that may not be seasonal.

The term asthma generally refers to a range of conditions, including those resulting from exposure to allergens, irritating gases, pollutants and chemical exposure as well as symptoms arising from cold or dry air exposure or exercise/physical activity.

The term inner ear disorders generally refers to conditions defined by hearing loss, tinnitus or vertigo that can have a number of causes including, but not limited to, noise and endolymphatic hydrops.

The term “tenosynovitis” generally refers to a range of such conditions, including those resulting from infective and non-infective causes. Non-infective causes include autoimmune disorders, overuse of affected tissues, and idiopathic in which there is no established cause for the condition.

The term “enthesitis” generally refers to a range of such conditions, including those resulting from infective and non-infective causes. Non-infective causes include injury, overuse, diseases such as ankylosing spondylitis and idiopathic in which there is no established cause for the condition.

The term “arthritis” generally refers to a range of such conditions, including those resulting from infective and non-infective causes. Non-infective causes include osteoarthritis, gout and autoimmune disorders such as rheumatoid arthritis, psoriatic arthritis, ankylosing spondylitis and systemic lupus erythematosus.

The term “subject” generally refers to a host animal to be treated using the compositions and methods described herein, such as, but not limited to humans and animals, including companion animals and livestock, such as dogs, cats, rabbits, birds, cattle, horses, monkeys, sheep, goats, reptiles, hamsters, guinea pigs and other animals, whether domesticated or not. Illustratively, subjects include host animals having or diagnosed with a skin or eye injury, a burn injury, allergic rhinitis/sinusitis or asthma, an inner ear disorder, including hearing loss, tinnitus or vertigo, tenosynovitis, tendinitis, enthesitis or arthritis.

The term “granulation tissue” generally refers to new connective tissue and microscopic blood vessels that form in a wound during the healing process. Granulation tissue typically grows from the sides and base of a wound and is able to fill wounds of almost any size. Examples of granulation tissue can be seen in a healing wound resulting from a surgical operation. Its histological appearance is characterized by proliferation of fibroblasts and new thin-walled, delicate capillaries (angiogenesis), infiltrated inflammatory cells in a loose extracellular matrix of proteins such as collagens.

The term “corneal opacification” is generally used when there is loss of transparency of the cornea due to a corneal opacity or scar that can prevent light from reaching the retina at the back of the eye. As a result, images can become distorted or blurred, leading to decreased or complete loss of vision. Transparency of the cornea depends on the correct arrangement of collagen fibrils in the cornea and the absence of edema. Alterations in the spacing and type of collagen fibrils and edema can be caused by burns or noxious chemicals that come into contact with the surface of the eye.

The terms “glucocorticoid” or “glucocorticosteroid” each generally refers to a class of corticosteroids, which are a class of steroid hormones. Glucocorticoids are corticosteroids that bind to the glucocorticoid receptor that is present in almost every vertebrate animal cell. Glucocorticoids are part of the stress response and play a role in feedback mechanisms in the immune system, which reduce certain aspects of immune function, such as inflammation. Because of this, they are often used to treat diseases caused by an overactive immune system (e.g., allergies, asthma, steroid-sensitive dermatoses, and autoimmune diseases). Clobetasol propionate is a highly selective and potent agonist for the glucocorticoid receptor.

The term “clobetasol propionate” generally refers to the following molecule.

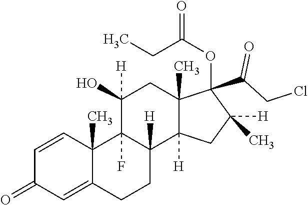

The term “nanomilling” generally refers to a process by which the particle size of an agent is reduced to nanometer dimensions via grinding using polymeric or ceramic media or other proprietary technologies. Nanomilling can be used for formulating poorly water-soluble APIs into small particles with a large surface that allows enhanced dissolution of the API.

The term “nanoparticle” generally refers to a small particle that typically ranges in mean particle size between 1 to 300 nanometers. Undetectable by the human eye as individual particles, nanoparticles can exhibit significantly different physical and chemical properties compared to correspondingly larger material counterparts.

The term “nanoparticle suspension” generally refers to a suspension that contains nanoparticle-sized particles of an active agent in a suspension. For example, glucocorticoids are hydrophobic and can be provided in the form of aqueous suspensions. However, the aqueous suspension of a glucocorticoid compound in particles larger than nanoparticles can be problematic as the larger steroid particles precipitate or cake over time, and thus a patient must shake a container before use to disperse an active component homogeneously in the liquid phase. Even with shaking, the larger particles in the suspension can easily aggregate or agglomerate to form clusters so that the particle diameter of the drug increases with time and the content uniformity of the suspension is lost. Because of these issues, it is difficult to obtain a uniform dispersion of larger particles and administer the active ingredient consistently. In addition, the reduced surface area of the larger particles reduces the dissolution rate of the API and hence its tissue penetration and therapeutic efficacy. However, the invention described herein does not have such aggregation or agglomeration issues.

As used herein, the particle size distribution of suspended nanoparticles in the formulation is determined using a dynamic light scattering method with a commercially available instrument such as Otsuka ELSZ-2000ZS particle size analyzer. The analyzer generates a histogram of the particle size distribution, with D₁₀, D₅₀, D₉₀ and mean particle size determined from the histogram.

The term “metered-dose spray” or “MDTS” generally refers to a device that delivers a drug to the surface of the skin, mucous membrane or airway and is absorbed into the tissue on a sustained basis. It works in a similar manner to a transdermal patch or topical gel. The drug is delivered by a device placed close to the skin or mucous membrane and triggered, causing it to release a light spray containing a proprietary formulation of the drug molecules to form an invisible drug depot. As it would be from a patch, the drug is then absorbed steadily for a predetermined amount of time.

The term “scar” generally refers to the connective tissue marking a spot where skin or other tissue has healed after it is damaged by, for example, a burn or other trauma. A scar is the body's natural way of healing and replacing lost or damaged skin or other tissue. A scar is usually composed of fibrotic tissue. A hypertrophic scar is an abnormal response to wound healing in which there is excessive connective tissue and collagen deposition within the original wound area, resulting in an abnormally raised scar. The term “keloid” generally refers to a type of abnormal scarring that extends beyond the initial wound area. In contrast to hypertrophic scars, keloid scars develop slowly and extend beyond the original wound area resulting in large abnormal scars that can be much larger than the original wound area. These types of scars can lead to significant, permanent disfigurement.

The term “contracture” generally refers to an abnormal occurrence that happens when a large area of skin is damaged and lost, resulting in a scar. The scar formation pulls the edges of the skin together, causing a tightening of the skin around the injured area. Contractures can also occur when there is chronic inflammation of a tendon, tendon sheath, tendon/ligament insertion or joint. A treatment option for contracture release is a surgery wherein the scar or chronically inflamed tissue is cut to release the tension. In some cases, there may be skin replacement with the donor tissues matching texture, color and pliability.

It is to be understood that in every instance disclosed herein, the recitation of a range for any variable is a description of the recited range itself, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

It is also to be understood that unless otherwise indicated the recitation of a numerical value necessarily reflects the relative precision of the numerical value. For example, the recitation of a number with a specified precision based on significant figures necessarily includes a range of values that would match that number after appropriate rounding. For example, the recitation of the number 1 with a single significant figure is understood to properly refer to a range of values from 0.5 to 1.4. Similarly, the recitation of the number 1.0 with two significant figures is understood to properly refer to a range of values from 0.95 to 1.04. The relative precision of the numerical value can be further indicated by modifying with the term “about” to indicate that the modified number has lower precision.

As used herein, the term “about” when used with numerical values or limits generally means that the number is approximate and that, as recited, it is understood to include a range of values. For example, a real number that is recited with a single significant figure, would by definition include a so-called rounding range; the number about 5 would at the very least include the range 4.5-5.4, as each of those values rounds to 5. The same is to be understood for real numbers expressed with additional significant figures, where the corresponding rounding range applies to the last significant figure. Integers are to be understood to at least include the values ±1 for single-digit numbers, ±10 for two-digit numbers, etc. Depending upon the context and the variable recited, the term “about” is also interpreted to contemplate a range based on a percentage of the recited number, such as about 5 construed to include 5±10% or in some instances 5±5%. Notwithstanding the foregoing, it is understood that the range of values, unless otherwise indicated, should not be interpreted to include a negative range for a positively recited number, and vice-versa. In addition, depending up on the context, the recited number, unless otherwise indicated, should not be interpreted to include a value of zero when used in conjunction with an added component.

All numerical designations, e.g., pH, temperature, time, concentration, and molecular weight, including ranges, are to be understood as approximations in accordance with common practice in the art.

Unless otherwise indicated, percentage (%) amounts of ingredients and components of the formulations described herein stated in units of % w/v refer to grams per deciliter (g/dL).

As used herein, the transitional phrase “consisting essentially of” means that the scope of the corresponding composition, unit dose, method or use is understood to encompass the specified compounds or recited steps, and those that do not materially affect the basic and novel characteristics of the invention described herein. For example, a method described herein that consists essentially of a single compound, or genus of compounds, is understood to represent a monotherapy for the recited disease. Though the monotherapy may include co-administration of one or more carriers, vehicles, diluents, adjuvants, excipients, and the like, and combinations thereof, and/or include co-administration of one or more additional active pharmaceutical ingredients, those latter additional active pharmaceutical ingredients are to be understood to be for treating diseases and/or symptoms distinct from treating the underlying conditions described herein, such as the treatment of the dermal or ocular burn injuries, abnormal wound healing, allergic rhinitis/sinusitis, asthma, inner ear disorders, tenosynovitis, tendinitis, enthesitis, or arthritis itself. Illustrative additional active pharmaceutical ingredients may include, for example, active ingredients for treating pain, accompanying bacterial infection, cough, congestion, and the like.

In embodiments that include a list of alternatives that may be included, either alone or in various combinations, it is to be understood that the list also describes each individual alternative as well as all possible subset lists of those alternatives.

Many known and useful compounds used in the formulation and the like can be found in Remington's Pharmaceutical Sciences (13^{th} Ed), Mack Publishing Company, Easton, PA—a standard reference for various types of administration. As used herein, the term “formulation(s)” refers to a combination of at least one active ingredient with one or more other ingredient, also commonly referred to as excipients, which may be independently active or inactive. It is to be understood that formulations described herein may or may not refer to a pharmaceutically acceptable composition for administration to humans or animals and may include compositions that are intermediates, such as for storage or research purposes.

Conventional topical preparations of corticosteroids have been proposed for the treatment of burns. (see, Brown, et al., Journal of Burn Care & Research, Volume 39, Issue suppl_1, April 2018, p. S239-S240). However, the use of such conventional preparations on damaged skin tissues can be painful and problematic. More serious tissue damage (e.g., third, and fourth degree burns) usually have significant loss of skin tissue and exudation of fluid. In these cases, conventional preparations may fail to adequately cover the damaged tissue area and ensure adequate penetration of the clobetasol propionate to the target areas without causing significant pain and discomfort. Furthermore, such conventional preparations formulations are intended for common skin ailments (e.g., allergic, inflammatory or autoimmune disorders). As such, those formulations may contain excipients, such as ethanol, that are included to improve the solubility of the corticosteroid, but which can cause severe pain when exposed to a burn wound, hinder drug exposure or healing of a burn wound, and/or are incompatible with certain treatments, including treatment of ocular burns.

Though certain glucocorticoids have been used to treat allergic rhinitis/sinusitis and asthma, it is believed herein that the formulations described herein, and/or the use of a glucocorticoid such as clobetasol propionate provide new treatment options. For example, the formulations described herein allow for smaller volumes of a spray or injection to be used. A smaller injection volume has the potential benefit of reducing post-injection pain when the injection site is limited, as is the case, for example, in a synovial sheath. In addition, the formulations described herein using glucocorticoids such as clobetasol propionate having a threshold level of activity may be applied directly to the tympanic membrane through the external ear canal as a spray or ear drop or injected intratympanically or applied to the nasal mucosal membranes to treat hearing loss, tinnitus or vertigo in cases where conventional formulations will be insufficient to improve symptoms.

### Nanoparticle Suspension of Clobetasol Propionate

The sterile suspension of nanoparticles of clobetasol propionate allows its delivery as a drop, uniform spray, mist, aerosol or as an injection. As a drop, spray or aerosol, it can be applied over the wound area, nasal cavity or inhaled into the lungs, or applied to the tympanic membrane in the ear canal to achieve even coverage while avoiding direct contact. The sterile suspension of nanoparticles of clobetasol propionate can also be administered as a drop onto the eye or into the nasal cavity or ear canal.

In other embodiments, suspension formulations of nanoparticles of clobetasol propionate are described. Certain suspensions described herein are optimized for treating complications of burns and can be evenly applied by spraying onto the injured area or by droplet application. Spray or aerosol application can cover a large surface area and avoid the pain associated with the required mechanical application of rubbing conventional ointments/lotions onto a burn injury to provide efficiency and comfort. The nanoparticle suspensions allow effective penetration of clobetasol propionate into the burn tissues thus presenting improved clinical efficacy and safety profiles compared to conventional formulations.

Certain suspension formulations described herein are optimized for treating abnormal wound healing and can be applied by spraying or dropping it onto the wound or by injection into the wound that is healing abnormally. The nanoparticle suspension allows effective penetration of clobetasol propionate into the tissues thus presenting superior clinical efficacy and tolerability profiles compared to conventional formulations.

Certain nanoparticle suspension formulations described herein are optimized for treating allergic rhinitis/sinusitis and asthma, inner ear disorders, specifically hearing loss, tinnitus or vertigo, tenosynovitis, tendinitis, enthesitis and arthritis and can be applied by spray, aerosol or drops onto the nasal and sinus mucosa or by inhaling into the lungs, or by application onto the tympanic membrane through the outer ear canal or by injection into the tympanic cavity or by injection into a synovial space, enthesis or joint. The nanoparticle suspension allows effective penetration of clobetasol propionate into the tissues thus presenting superior clinical efficacy and tolerability profiles compared to conventional formulations.

The nanoparticle suspension formulations can be produced by dispersing nanomilled clobetasol propionate optionally with a mixture of one or more excipients in an aqueous formulation. The nanomilling process allows clobetasol propionate to be formulated in a suspension with a mean particle size distribution below 250 nm range so that individual particles are not visible to the naked eye and the formulation appears as an opalescent liquid, and feels smooth and comfortable when applied to the skin, eyes or mucous membranes (eg., nasal or pulmonary) or injected into the tympanic cavity or synovial space, enthesis or joint or scar. Suspensions of clobetasol propionate nanoparticles described herein permit effective penetration of clobetasol propionate from the high surface area nanoparticles into tissues upon surface application or injection. Nanoparticle formulations described herein are designed to optimally deliver therapeutic amounts of clobetasol propionate to target tissues. In addition, such aqueous spray formulations will be compatible with damaged tissues that are exuding fluid when ointments, emulsions and/or creams, cannot be used. As an injection, the suspensions of nanoparticles of clobetasol propionate described herein may allow a more concentrated amount of drug to be injected in a small volume and may provide prolonged duration of action.

### Composition of the Spray or Injectable Formulation of Nanoparticle Suspension

In another embodiment, transdermal, transepithelial, transmembrane, transmucosal and transcorneal sprays, drops or aerosols are described herein. The sprays, drops or aerosols can be applied to the skin, eye, nasal cavity and sinus membranes or respiratory airways to treat ailments or injuries, without the negative effects of conventional ointments/lotions.

In another embodiment, injectable formulations are described herein. The injectable formulations can be injected into tendons/ligaments, synovial spaces or joints to treat ailments or injuries, without the negative effects of conventional ointments/lotions.

In another embodiment, a nanoparticle suspension formulation of spray, drop, mist, aerosol or injection of clobetasol propionate is prepared as a sterile liquid that is adapted to be filled into a sterile spray bottle, eye-drop bottle, aerosol device, or inhaler; or into an injectable vial or ampule, or into a pre-filled syringe for ease of use.

In another embodiment, a topical spray, drop, aerosol or injection formulation of a suspension of clobetasol propionate nanoparticles contains concentrations of clobetasol propionate ranging from about 0.01% to about 0.25% w/v inclusive, or from about 0.025% to about 0.15% w/v inclusive. In another embodiment, the concentration is about 0.025%, about 0.05%, about 0.075%, about 0.1%, about 0.125%, or about 0.15% w/v.

In another embodiment, a topical spray, drop, aerosol or injection formulation of a suspension of clobetasol propionate nanoparticles contains sodium chloride in a concentration ranging from about 0.05% to about 3.0% w/v, or from about 0.15% to about 1.5% w/v. In another embodiment, the concentration is about 0.6%, about 0.8%, or about 1.2% w/v.

In another embodiment, a topical spray, drop, aerosol or injection formulation of a suspension of clobetasol propionate nanoparticles contains lecithin in a concentration about 0.01% to about 0.5% w/v, or from about 0.025% to about 0.25% w/v. In another embodiment, the concentration is about 0.025, about 0.05%, about 0.075%, about 0.1%, about 0.15%, about 0.2% or about 0.25% w/v.

In another embodiment the lecithin is hydrogenated soybean lecithin. In another embodiment, the lecithin is from an egg yolk, marine sources (including algae and kelp), milk, rapeseed, cotton seed and sunflower oil. In another embodiment, the lecithin is a hydrolyzed lecithin.

In another embodiment, a topical spray, drop, aerosol or injection formulation of a suspension of clobetasol propionate nanoparticles contains citric acid in a concentration ranging from about 0.001% to about 0.4% w/v, or from about 0.001% to about 0.012% w/v. In another embodiment, the concentration is about 0.002%, about 0.004%, about 0.006%, about 0.008%, or about 0.01% w/V.

In another embodiment, a topical spray, drop, aerosol or injection formulation of a suspension of clobetasol propionate nanoparticles contains glycerin in a concentration ranging from about 0.04% to about 0.6% w/v, or from about 0.08% to about 0.32%. In another embodiment, the concentration is about 0.08%, about 0.12%, about 0.16%, about 0.20%, about 0.24%, about 0.28% or about 0.32% w/v.

In another embodiment, a topical spray, drop, aerosol or injection formulation of a suspension of clobetasol propionate nanoparticles contains a non-ionic surfactant in a concentration ranging from about 0.002% to about 0.05% w/v, or from about 0.0025% to about 0.025%. In another embodiment, the concentration is about 0.0025%, about 0.005%, about 0.01%, about 0.015%, about 0.02% or about 0.025% w/V.

In another embodiment, the non-ionic surfactant is Poloxamer 407. In another embodiment, the non-ionic surfactant is polysorbate, polysorbate 20, polysorbate 80, sorbitan, sorbitan monolaurate, sorbitan monostearate, sorbitan tristearate, stearyl alcohol, surfactin, or any combination thereof, including a combination with Poloxamer 407.

In another embodiment, a topical spray, drop, aerosol or injection formulation of a suspension of clobetasol propionate nanoparticles contains a thickener in a concentration ranging from about 0.01% to about 2% w/v, or from about 0.025% to about 0.25% w/v. In another embodiment, the concentration is about 0.025%, about 0.05%, about 0.075%, about 0.1%, about 0.15%, about 0.2% or about 0.25% w/v.

In another embodiment, a thickener is polyvinyl alcohol (PVA), agar, albumin, an alginate, casein, cetyl alcohol, cholic acid, desoxycholic acid, diacetyl tartaric acid esters, egg yolk glycerol, gums, Irish moss (carrageenan), a monoglyceride, a diglyceride, monostearate, sucrose esters, stearoyl lactylates, propylene glycol (including their esters), and any combination thereof.

In another embodiment, a topical spray, drop, aerosol or injection formulation of a suspension of clobetasol propionate nanoparticles contains boric acid in a concentration ranging from about 0.0% to about 0.5% w/v, more specifically from about 0% to about 0.25% w/v. In another embodiment, the concentration is about 0.0%, about 0.05%, about 0.1%, about 0.15%, about 0.2%, or about 0.25% w/v.

In another embodiment, a topical spray, drop, aerosol or injection formulation of a suspension of clobetasol propionate nanoparticles contains edetate disodium dihydrate (EDTA) in a concentration ranging from about 0.0% to about 0.3% w/v, or from about 0.0% to about 0.15% w/v. In another embodiment, the concentration is about 0.0%, about 0.01%, about 0.02%, about 0.03%, about 0.04%, or about 0.05% w/V.

It has been unexpectedly discovered herein that combinations of boric acid and EDTA improve tissue penetration by the formulations described herein. In another embodiment, aqueous suspensions of nanoparticles described herein that include boric acid and EDTA provide higher tissue concentrations (e.g., peak concentration Cₘₐₓ) of the active drug molecule, such as clobetasol propionate, at equivalent administered doses. In another embodiment, aqueous suspensions of nanoparticles described herein that include boric acid and EDTA do not exhibit a loss of long therapeutic exposure to the active drug molecules, such as clobetasol propionate, and maintain significant half-life in the tissue.

The suspension formulations described herein optionally include a preservative. Benzalkonium chlorides (BACs) are antibacterial agents with an established history of use in pharmaceutical preparations to maintain sterility of the product. In conventional formulations, the BAC level is generally required to be at about 0.1% w/v to be effective as a preservative. However, such high concentrations of BACs often cause irritation or inflammation. It has been surprisingly discovered herein that the BAC level can be minimized to avoid irritation and inflammation when combined with certain additional excipients, such as boric acid and EDTA. Antimicrobial effectiveness is maintained with the lower level of BACs when in combination with boric acid and EDTA. It has been unexpectedly discovered that BACs reduced below 0.006% to about 0.004%, or about 0.003%, or less when in combination with boric acid and EDTA maintain antimicrobial effectiveness and are effective preservatives of the formulations described herein.

In another embodiment, a topical spray, drop, aerosol or injection formulation of a suspension of clobetasol propionate nanoparticles contains benzalkonium chloride in a concentration ranging from about 0.0% to about 0.03% w/v, or from about 0.0% to about 0.01% w/v. In another embodiment, the concentration is about 0.0%, about 0.002%, about 0.003%, about 0.0036%, about 0.004%, about 0.005%, about 0.006%, about 0.007%, about 0.008%, about 0.009%, or about 0.01% w/v.

Additional preservatives may be included at concentrations effective to inhibit microbial growth, ultraviolet light and/or oxygen-induced breakdown of composition components, and the like. When one or more preservatives are included, each, or the combination thereof, can range in concentration from about 0.01% w/v to about 2% w/v. In another embodiment, a preservative can include phenol, benzyl alcohol, phenoxyethanol, propylparaben, methylparaben, benzoic acid, sorbic acid, bronidiol, or propylene glycol, or a combination thereof.

In another embodiment, a topical spray, drop, aerosol or injection formulation of a suspension of clobetasol propionate nanoparticles contains methylcellulose in a concentration ranging from about 0.00% to about 3.0% w/v, or from about 0.0% to about 2.0%, w/v. In another embodiment, the concentration is about 0.0%, about 0.1%, about 0.2%, about 0.5%, about 0.75%, about 1.0%, about 1.5% or about 2.0% w/v.

It has been unexpectedly discovered herein that a higher concentration of methyl cellulose improves drug molecule retention at the local target tissue site by the formulations described herein. In another embodiment, aqueous suspensions of nanoparticles described herein that include higher concentration of methyl cellulose provide higher tissue concentrations (e.g., peak concentration Cₘₐₓ) of the active drug molecule, such as clobetasol propionate, at equivalent administered doses. In another embodiment, aqueous suspensions of nanoparticles described herein that include higher concentration of methyl cellulose provide longer therapeutic exposure to the active drug molecule, such as clobetasol propionate, as shown by a longer half-life in the target tissue.

In another embodiment, a topical spray, drop, aerosol or injection formulation of suspension of clobetasol propionate nanoparticles contains sodium citrate, such as trisodium citrate, in a concentration ranging from about 0.01% to about 0.8% w/v, in order to adjust the pH of the nanosuspension to the appropriate range, such as about 4.5 to about 6.5.

The formulation described herein can also include more than one additional therapeutic compounds as desired for the indication being treated (e.g., a skin or ocular burn wound). Additional therapeutic compounds generally exhibit complementary activities that do not adversely affect the activity of the other components. An illustrative therapeutic compound that can be used as part of a combination to treat a burn wound, infective tenosynovitis or infective arthritis is an antimicrobial. Other therapeutics that can be used as a combination therapeutic compound to treat a burn wound are silver sulfadiazine, penicillin or a penicillin derivative such as amoxicillin, including when combined with a beta-lactamase inhibitor such as amoxicillin-clavulanate, a cephalosporin such as cephalexin, a fluoroquinolone such as ciprofloxacin, a tetracycline such as doxycycline, a macrolide such as erythromycin, an aminoglycoside such as gentamicin, an oxazolidinone such as linezolid, a glycopeptide such as vancomycin, daptomycin, fusidic acid, clindamycin, trimethoprim-sulfamethoxazole, metronidazole, daptomycin, linezolid, rifampin, bacitracin, neomycin, polymyxin B or E, nystatin, amphotericin B, an echinocandin such as caspofungin, a triazole such as fluconazole, or combination thereof. Other therapeutics that can be used to treat ocular burns include nonsteroidal anti-inflammatory drugs or antimicrobials and combinations thereof.

In another embodiment, antimicrobials for use to treat a skin or ocular burn include, but are not limited to, silver sulfadiazine, penicillin or a penicillin derivative such as amoxicillin including when combined with a beta-lactamase inhibitor such as amoxicillin-clavulanate, a cephalosporin such as cephalexin, a fluoroquinolone such as ciprofloxacin, a tetracycline such as doxycycline, a macrolide such as erythromycin, an aminoglycoside such as gentamicin, an oxazolidinone such as linezolid, a glycopeptide such as vancomycin, daptomycin, fusidic acid, clindamycin, trimethoprim-sulfamethoxazole, metronidazole, daptomycin, linezolid, rifampin, bacitracin, neomycin, polymyxin B or E, nystatin, amphotericin B, an echinocandin such as caspofungin, a triazole such as fluconazole, or combination thereof.

In another embodiment, antimicrobials for use to treat allergic rhinitis/sinusitis and asthma with secondary infection include but are not limited to, penicillin or a penicillin derivative such as amoxicillin including when combined with a beta-lactamase inhibitor such as amoxicillin-clavulanate, a cephalosporin such as cephalexin, a fluoroquinolone such as levofloxacin, a tetracycline such as doxycycline, a macrolide such as erythromycin, clindamycin, trimethoprim-sulfamethoxazole, or combination thereof, doxycycline, trimethoprim-sulfamethoxazole, ciprofloxacin, penicillin or combination thereof.

In another embodiment, antimicrobials for use to treat an infected tendon or joint include but are not limited to, penicillin or a penicillin derivative such as dicloxacillin including when combined with a beta-lactamase inhibitor such as amoxicillin-clavulanate, a cephalosporin such as cephalexin, a fluoroquinolone such as ciprofloxacin, a tetracycline such as doxycycline, a carbapenem such as meropenem including when combined with a beta-lactamase inhibitor such as meropenem-vaborbactam, an aminoglycoside such as gentamicin, an oxazolidinone such as linezolid, a glycopeptide such as vancomycin, daptomycin, fucidic acid, clindamycin, trimethoprim-sulfamethoxazole, rifampin or combination thereof.

In another embodiment, aqueous suspensions described optionally include a local anesthetic, such as lidocaine, and/or a suitable antimicrobial can be incorporated in the formulation to further reduce pain, provide comfort and reduce the risk of infection. Anesthetics that can be used as part of a combination with clobetasol propionate include, but are not limited to, lidocaine, pramoxine, phenol, prilocaine, benzocaine, dibucaine, menthol, methyl salicylate, zinc acetate, camphor, tetracaine, pentafluoropropane, tetrafluoroethane, or any combination thereof.

The suspension comprising nanoparticles of clobetasol propionate disclosed herein may further include a pharmaceutically acceptable carrier, excipient, or diluent. Illustrative excipients are those having the “Generally Regarded As Safe” (GRAS) designation. For example, preservatives like antioxidants e.g., ascorbic acid, sorbic acid or α-lipoic acid and antimicrobial agents may be included. Other components apart from therapeutically active ingredients and components that are the primary effectors of dermal, ocular, inner ear, epithelial or synovial penetration, or may include those provided for aesthetic purposes such as menthol or other aromatics, and components that affect the physical state of the composition such as lubricants in the case of intraarticular or synovial space injections. These ingredients may be present in very small or large percentages of the compositions.

The disclosed suspension comprising nanoparticles of clobetasol propionate may be formulated into a variety of dosage forms in combination with the aforementioned pharmaceutically acceptable carriers. For example, for topical administration by use of a spray (including an aerosol spray for inhalation or spraying) or droplets, the pharmaceutical composition may be formulated into a liquid form that can be sprayed onto a burn wound of a subject, or for example, applied as a droplet to the surface of the skin or eye, or into the ear or nose, or for example, for injection use the pharmaceutical composition may be formulated into a liquid form that can be injected intra-tympanically, or into a keloid or hypertrophic scar, or into an inflamed synovial space, tendon/ligament insertion or joint of a subject.

The formulations described herein can be sterile. Sterile formulations are illustratively prepared by filtration through sterile filtration membranes, prior to, or following, preparation of a spray, drop, liquid or injection formulation or other methods known in the art, including without limitation, pasteurization and gamma irradiation.

A formulation in accordance with the subject matter described herein may be a topical dosage form packaged in, for example, a multi-use or single-use package, including for example, a tube, a pump, a container or bottle, a vial, a jar, a can, a cassette, a packet, or an injectable form packaged in, for example, a multi-use or single-use package or in pre-filled syringes. Single dosage kits and packages containing a once per day amount or other appropriate amount of the topical delivery formulation or injectable may be prepared. Single dose, unit dose, and once-daily disposable containers of the formulation are also provided.

In certain embodiments, the therapeutic such as a suspension comprising nanoparticles of clobetasol propionate to treat a dermal or ocular burn, abnormal wound healing, allergic rhinitis/sinusitis and asthma, inner ear disorder, specifically hearing loss, tinnitus or vertigo, tenosynovitis, tendinitis, enthesitis or arthritis is incorporated into a kit. In another embodiment, a kit can comprise, without limitation, one or more single or multi-chambered spray bottles, inhalers of any form, dropper bottles or injection vials/ampoules or prefilled syringes for administering one or more therapeutic compound(s) (including clobetasol propionate) described herein. In various embodiments, the kit can comprise pharmaceutical composition for topical or injection administration, including through the application of a drop, spray, aerosol, mist or injection that comprises one or more therapeutic compound(s), wherein at least one is clobetasol propionate for administration to a subject. In this regard, a therapeutic compound(s) can be disposed therein by application of a spray or drop to the skin, eye, nasal and sinus mucous membranes, by inhalation into the lungs, or by application to the tympanic membrane or by intra-tympanic injection or by injection into hypertrophic scars, keloid, synovial space, tendon/ligament insertion or joint. In all of these embodiments and others, the kits can contain one or more spray bottles, inhalers of any form, dropper bottles or injection vials/ampoules or prefilled syringes in accordance with any of the foregoing, instructions and/or packing material.

### Methods of Use

In another embodiment, the period of administration of a therapeutic compound, such as an aqueous suspension comprising nanoparticles of clobetasol propionate to treat a dermal or ocular burn wound, abnormal wound healing, allergic rhinitis/sinusitis and asthma, inner ear disorder, specifically hearing loss, tinnitus or vertigo, tenosynovitis, tendinitis, enthesitis or arthritis including clobetasol propionate is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period during which administration is paused or stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In another embodiment, the dose of the therapeutic compound, a suspension comprising nanoparticles of clobetasol propionate, may be administered multiple times a day, daily, semi-weekly, weekly, bi-weekly, or monthly. The initial dose or doses may be larger or smaller than subsequent doses.

In another embodiment, the therapeutically effective amount of a suspension comprising nanoparticles of clobetasol propionate to treat a subject suffering from a burn wound disclosed herein reduces the size or severity of the burn wound site by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, the therapeutically effective amount of a suspension comprising nanoparticles of clobetasol propionate to treat a subject suffering from an ocular burn wound disclosed herein improves vision by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, the therapeutically effective amount of a suspension comprising nanoparticles of clobetasol propionate to treat a subject suffering from abnormal wound healing disclosed herein reduces the size or severity of the hypertrophic scarring or keloid by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, the therapeutically effective amount of a suspension comprising nanoparticles of clobetasol propionate to treat a subject suffering from allergic rhinitis/sinusitis or asthma disclosed herein reduces the mucous membrane or airway inflammation by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, the therapeutically effective amount of a suspension comprising nanoparticles of clobetasol propionate to treat a subject suffering from an inner ear disorder disclosed herein improves the symptoms of hearing loss, tinnitus or vertigo by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, the therapeutically effective amount of a suspension comprising nanoparticles of clobetasol propionate to treat a subject suffering from tenosynovitis, tendinitis, enthesitis or arthritis disclosed herein reduces the tendon, ligament or articular inflammation by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, symptoms or complications associated with a dermal or ocular burn injury, abnormal wound healing, allergic rhinitis/sinusitis and asthma, inner ear disorders, tenosynovitis, tendinitis, enthesitis or arthritis described herein (e.g., hypertrophic granulation and unstable scar, keloid formulation, impaired vision, nasal stuffiness and discharge, airway inflammation, hearing loss, tinnitus, vertigo, joint pain, tendon insertion pain or “trigger finger”) are reduced following administration of a formulation by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, application of the formulation described herein decreases healing and recovery time of a burn injury, abnormal wound healing, allergic rhinitis/sinusitis and asthma, inner ear disorder, specifically hearing loss, tinnitus or vertigo, tenosynovitis, tendinitis, enthesitis or arthritis by 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, application of the formulation described herein decreases pain and/or discomfort caused by a burn injury, tenosynovitis, tendinitis, enthesitis or arthritis by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, application of the formulation described herein improves mucosal edema and inflammation and reduces bronchoconstriction caused by allergic rhinitis/sinusitis or asthma by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In another embodiment, a suspension comprising nanoparticles of clobetasol propionate reduces the frequency of a side effect or complication associated with a burn wound, abnormal wound healing, allergic rhinitis/sinusitis and asthma, inner ear disorder, specifically hearing loss, tinnitus or vertigo, tenosynovitis, tendinitis, enthesitis or arthritis over a given time period. In aspects of this embodiment, a suspension comprising nanoparticles of clobetasol propionate reduces the frequency of a side effect or complication associated with a burn wound, abnormal wound healing, allergic rhinitis/sinusitis and asthma, inner ear disorder, specifically hearing loss, tinnitus or vertigo, tenosynovitis, tendinitis, enthesitis or arthritis over a given time period by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In another embodiment, the spray or droplet of the suspension of nanoparticles of clobetasol propionate is applied directly to an area of skin, eye, nasal and sinus mucous membranes, airways in the lungs, or the tympanic membrane that is affected by an ailment and/or injury. In applying a formulation of the invention disclosed herein, a formulation itself is illustratively administered by droplet to the eye or nose, or sprayed on the skin, eye, nasal and sinus mucous membrane, inhaled into the lung, or applied to the tympanic membrane by, for example, using a spray or dropper bottle or another appropriate device. The spray bottle can be a pump spray bottle, a spray mist bottle, an inhaler of any form, an aerosol spray bottle, an atomizer bottle, a metered-dose device etc. The dropper bottle can deliver drops of predefined approximate volume such as approximately 50 μL per administration. The amount of formulation used is typically sufficient to cover a desired surface area. In some embodiments, a protective cover is placed over the formulation once it is applied and left in place for a suitable amount of time (e.g., 5 minutes, 10 minutes, 20 minutes or more; in some embodiments an hour or two or longer). The protective cover can simply be a bandage including a bandage supplied with a cover that is impermeable to moisture. For ocular burns, the protective cover can simply be a bandage to protect the external eye tissues and reduce the risk of mechanical damage or infection. For dermal applications, this essentially locks in the contact of a formulation to the skin and prevents distortion of a formulation by evaporation or the distribution of the therapeutic agent to other parts of the body or to other persons. It is also possible to apply the formulation to the skin using standard procedures for application such as a brush, a syringe, a gauze pad, a dropper, or any convenient applicator (e.g., for smaller areas). More complex application methods, including the use of delivery devices, may also be used, but are not required. External reservoirs of the formulations for extended administration may also be employed.

In another embodiment, the suspension of nanoparticles of clobetasol propionate is injected intra-tympanically or into the synovial space, tendon/ligament insertion or joint, or keloid or a hypertrophic scar that is affected by an ailment and/or injury. In applying a formulation disclosed herein, a formulation itself is illustratively injected, for example, using a syringe and needle. The amount of formulation used is typically minimal and sufficient to treat the ailment and/or injury. More complex application methods, including the use of delivery devices, may also be used, but are not required. External reservoirs of the formulations for extended administration may also be employed. Without being bound by theory, it is believed herein that potent glucocorticoids, such as clobetasol propionate, reduce pathological scarring by decreasing inflammation and activation of fibroblasts to limit the synthesis of proteins such as collagen that characterize scar tissue, and ultimately lead to the resolution of hypertrophic scars or keloids.

A formulation can be applied in a single, one-time application, or more often as needed, such as, for example, at a frequency of once per day to hourly if needed. The formulations disclosed herein can be topically administered once or more per day for a period of time from one week to four weeks, from one week to two weeks, for one week, for two weeks, for three weeks, for four weeks, or for four weeks or more. In some cases, it may also be desirable to continue treatment indefinitely or as symptoms persist. A suitable administration for a formulation is, for example once, twice, three, four times daily, six times daily, 8 times daily, or hourly if needed. The presently described formulations can be injected once or more per day for a period of time from one week to four weeks, from one week to two weeks, for one week, for two weeks, for three weeks, for four weeks, or for four weeks or more.

Compositions in accordance with embodiments described herein have desirable properties, such as desirable solubility, viscosity, sprayability, spreadability, tonicity, injectability, syringeability, compatibility, homogeneity, resuspendability, sterility, and stability.

In another embodiment, the pH of the formulation described is in the range of about 2 to about 8, or from pH about 4.5 to about 6.5.

### Examples

It is to be understood that both the foregoing description and the following working examples are exemplary and explanatory and are intended to provide further understanding of the nature and scope of the invention described herein. The following non-limiting examples are to be understood as a representative subset of working embodiments of the invention, and should not be construed to limit any of the embodiments described herein, including those pertaining to the type and amounts of components of the formulation and/or methods and uses thereof.

### Example 1. Preparation of the Suspension Formulations of Nanoparticles of Clobetasol Propionate

Suspension formulations of nanoparticles of clobetasol propionate are prepared by grinding clobetasol propionate drug particles with milling agents as illustratively described in U.S. Pat. Nos. 8,226,983, 10,588,913, and 11,376,262, and US Patent Application Publication Nos. US 2018/0117064 and US 2020/0129526 to form a clobetasol propionate nanomixture. Subsequently, the nanomixture is dispersed with various excipients as illustratively described in Tables 1A, 1B, and 2 in an aqueous medium.

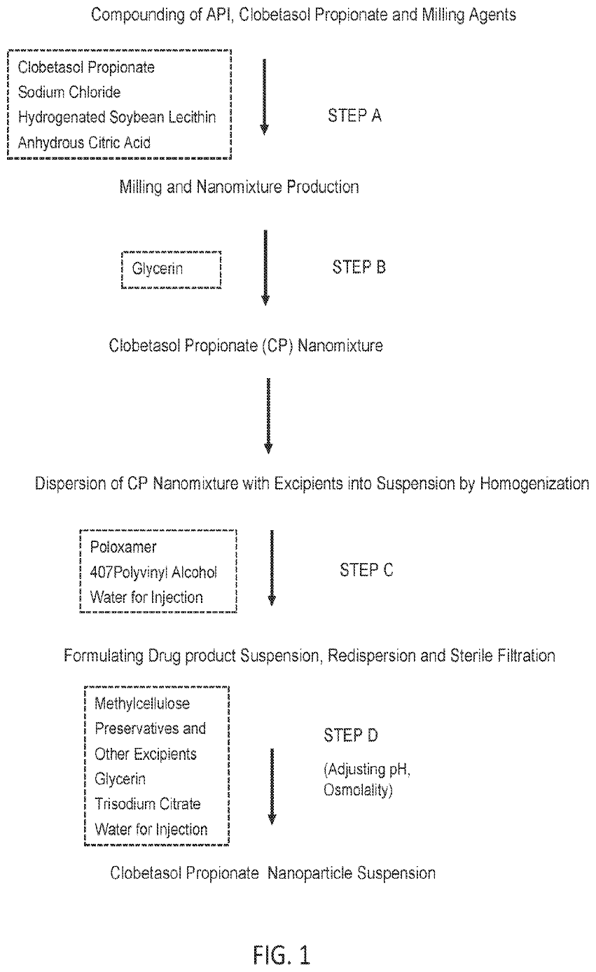

FIG. 1 shows a summary flowchart of steps employed to produce illustrative nanoparticle suspensions of clobetasol propionate. Step A involves compounding the active pharmaceutical ingredient (API) with milling agents. Illustrative milling agents include sodium chloride, hydrogenated soybean lecithin, and anhydrous citric acid. Step B involves milling the compounded mixture with glycerin to produce the milled clobetasol propionate nanomixture with particle size distribution D₉₀ less than 250 nm.

Step C involves dispersion of the clobetasol propionate nanomixture with Poloxamer 407 and polyvinyl alcohol into suspension using a high shear-force homogenizer to make the nanosuspension with particle size distribution D₉₀ less than 250 nm. In Step D, the mixture is formulated and mixed with other excipients including methylcellulose, preservatives (boric acid, EDTA, and benzalkonium chloride) followed by adjusting the pH with sodium citrate, such as trisodium citrate, and tonicity with glycerin and q.s. with water for injection (WFI). In Step E, the formulated suspension undergoes final redispersion before sterile filtration to produce the final aqueous suspension of nanoparticles of clobetasol propionate.

The nanomilling process produces the suspension of clobetasol propionate nanoparticles having a mean particle size distribution in the range of approximately 120-200 nm, and appears as an opalescent liquid without visible particles to the naked eye, and feels smooth and comfortable when applied to the skin or mucous membranes. It can be delivered as a sterile spray onto a large area of burned wound or other mucous membranes, or as a drop to a more limited area of skin, eye, nose, mucous membrane, or tympanic membrane, or as an injection into a tympanic cavity, keloid or scar tissue, synovial space, tendon/ligament insertion, joint or scar. The clobetasol propionate nanoparticles in the formulation effectively penetrate into tissues upon surface application or injection to deliver therapeutically effective concentrations of clobetasol propionate to the target tissues within a skin or ocular burn wound, nasal cavity, lungs, ear canal, inner ear, synovial space, tendon/ligament insertion, joint or scar. The aqueous sprays and drops are conducive to use with tissues that are exuding fluid such as a burn wound or inflamed nasal and sinus mucous membranes and respiratory airway.

Illustrative formulation compositions are shown in Tables 1A, 1B and 2.

TABLE 1A
Illustrative Compositions of Suspension Formulations
of Clobetasol Propionate Nanoparticles

| Component | % w/v |
|---|---|
| Clobetasol Propionate | 0.05-0.25 |
| Sodium Chloride | 0.15-1.5 |
| Hydrogenated Soybean Lecithin | 0.1-0.5 |
| Anhydrous Citric Acid | 0.002-0.1 |
| Glycerin | 0.04-0.6 |
| Poloxamer 407 | 0.002-0.2 |
| Polyvinyl Alcohol (PVA) | 0.1-1.0 |
| Boric Acid | 0.0-0.5 |
| Edetate Disodium Dihydrate | 0.0-0.3 |
| Benzalkonium Chloride | 0.00-0.03 |
| Methylcellulose | 0.2-2.0 |
| Trisodium Citrate | QS to adjust pH to 4.5-6.5 |
| Water for injection | QS to 100% |

% expressed as w/v (weight/volume), e.g. g/dL.
QS = quantum satis

TABLE 1B
Illustrative Compositions of Suspension Formulations
of Clobetasol Propionate Nanoparticles

| Component | % w/v |
|---|---|
| Clobetasol Propionate | 0.01-0.15 |
| Sodium Chloride | 0.15-1.5 |
| Hydrogenated Soybean Lecithin | 0.01-0.15 |
| Anhydrous Citric Acid | 0.001-0.02 |
| Glycerin | 0.02-0.30 |
| Poloxamer 407 | 0.005-0.05 |
| Polyvinyl Alcohol (PVA) | 0.05-0.3 |
| Boric Acid | 0.0-0.5 |
| Edetate Disodium Dihydrate | 0.00-0.10 |
| Benzalkonium Chloride | 0.00-0.03 |
| Methylcellulose | 0.1-1.0 |
| Trisodium Citrate | QS to adjust pH to 4.5-6.5 |
| Water for injection | QS to 100% |

% expressed as w/v (weight/volume), e.g. g/dL.
QS = quantum satis

TABLE 2
Illustrative Compositions of Suspension Formulations
of Clobetasol Propionate Nanoparticles

| | 0.1% | 0.05% |
|---|---|---|
| | Nanosuspension | Nanosuspension |
| | (CPN 0.1%) | (CPN 0.05%) |
| Component | % (w/v) | % (w/v) |
| Clobetasol propionate | 0.1% | 0.05% |
| Sodium Chloride | 1.2% | 0.59% |
| Hydrogenated Soybean Lecithin | 0.11% | 0.054% |
| Anhydrous Citric Acid | 0.009% | 0.004% |
| Glycerin | 0.16% | 0.08% |
| Poloxamer 407 | 0.005% | 0.005% |
| Polyvinyl Alcohol | 0.1% | 0.1% |
| Boric Acid | 0.25% | 0.25% |
| Edetate Disodium | 0.05% | 0.05% |
| Benzalkonium chloride | 0.0036% | 0.0036% |
| Methylcellulose | 0.5% | 0.5% |
| Glycerin | — | q.s. to 250-350 |
| | | mOsm/kg |
| Trisodium Citrate | q.s. to pH 6.0 | q.s. to pH 6.0 |
| Water for Injection | q.s. to 100% | q.s. to 100% |

% expressed as w/v (weight/volume), e.g. g/dL.
q.s. = quantum satis

### Example 2. Treatment of Ocular Burn

The efficacy of clobetasol propionate nanosuspension 0.1% (CPN 0.1%) was assessed in an ocular alkali burn model in New Zealand White rabbits (n=5 CPN 0.1% group; n=5 vehicle group). Baseline slit lamp ophthalmic examinations, anterior optical coherence tomography (OCT), and corneal surface fluorescein staining was performed to confirm lack of ocular abnormalities or injuries. Ophthalmic examinations were scored according to a modified McDonald-Shadduck Scoring System, and a score of zero for all parameters was required for study inclusion.

On day 1, animals were anesthetized with ketamine and xylazine, and a topical anesthetic was applied to the study eye. To produce the ocular alkali burn, a round filter paper (No. 1 Whatman; ~5 mm in diameter) was soaked with 1 M NaOH for ~10 seconds and then placed on the center of the cornea of the study eye for ~20 seconds. Immediately after removing the filter paper, the ocular surface was rinsed with ~30 ml of balanced salt solution. Thirty minutes after ocular injury, the study eye was treated with one drop (~50 μL) of CPN 0.1% or vehicle (n=5 CPN 0.1% group; n=5 vehicle group). These treatments were continued four times per day (separated by 2 h) on days 1-7 of the study. Antibiotic eye drops were applied to the study eye three times a day on days 1-3 to prevent infection. Ocular examinations, slit lamp photographs, OCT measurements, and surface fluorescein staining were performed on day 1 immediately after the alkali burn wounds were rinsed and on days 3 and 7 prior to dosing. Slit lamp examinations assessed corneal opacity, corneal area of injury, corneal vascularization, and conjunctival discharge, congestion, and swelling. Anterior OCT and fluorescein staining imaging were performed using a Spectralis® imaging platform and a Heidelberg Retina Angiograph-OCT device.

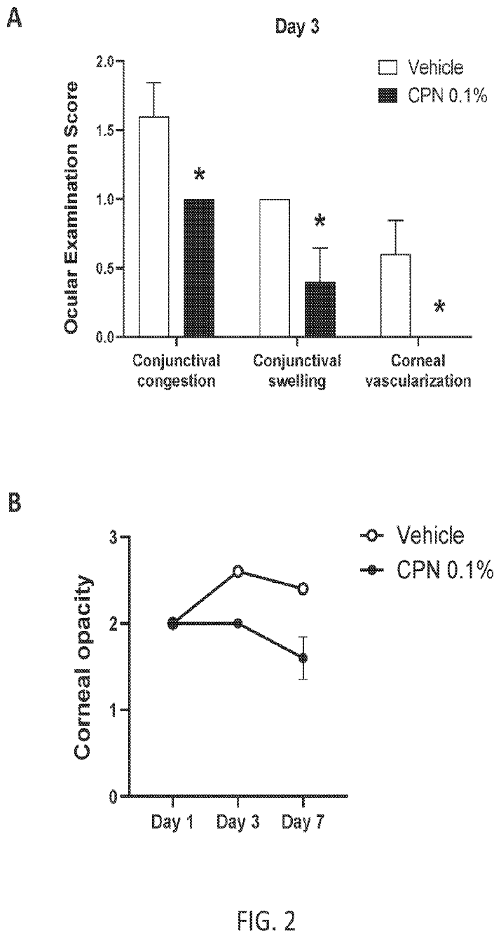

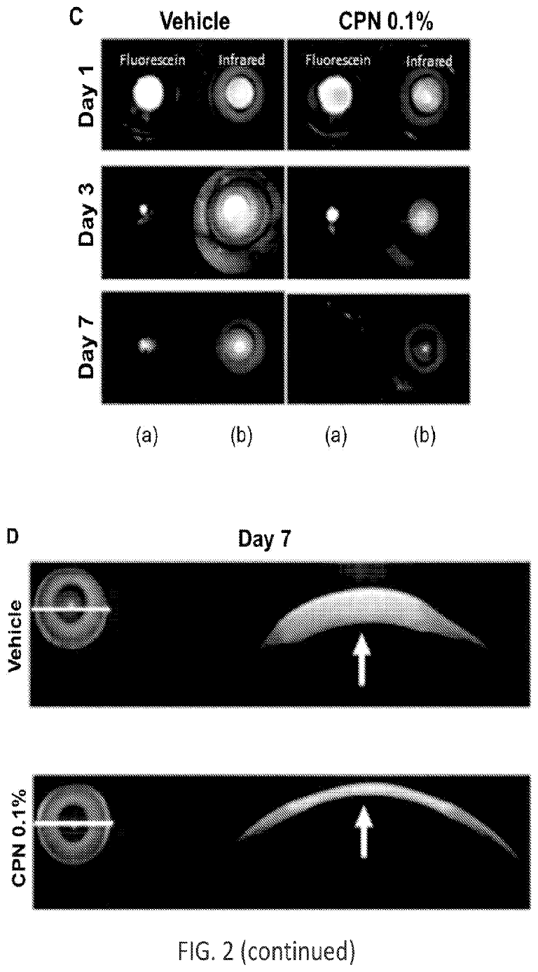

The results are shown in FIG. 2. By day 3, CPN 0.1% treatment significantly and rapidly reduced clinical scores of inflammation, including conjunctival congestion and swelling, and corneal neovascularization compared to vehicle (Student's unpaired t-test, p<0.05) (Panel A). CPN 0.1% also rapidly reduced clinical scores of corneal opacity and stromal haze by day 3 compared to vehicle, which was maintained through day 7 [two-way ANOVA, F (1, 8)=12.25, p=0.008] (Panel B). By day 7, surface fluorescein staining showed that CPN 0.1% treatment resulted in faster healing of the corneal epithelium compared to vehicle (Panel C). Panel C shows representative images of fluorescein staining (a) of the central corneal epithelial defects and corresponding infrared images (b) of the eye indicating the location of the pupil (central cornea) and iris. The magnitude of fluorescein staining defines the extent of epithelial damage on the corneal surface. By day 7, the CPN 0.1% group had little to no fluorescein staining present, while the vehicle treated eye still had significant fluorescein staining indicating continued damage to the corneal epithelium. By day 7, anterior OCT imaging showed corneal thickness (edema) and corneal stromal haze were markedly reduced, compared to vehicle (Panel D). Panel D shows representative cross-sectional OCT images of injured rabbit corneas on day 7 (the planes of the cross-sections are shown by the horizontal white lines in the images on the left side). The markedly reduced thickness of the cornea OCT image in the CPN 0.1% group (bottom image, arrow) indicates significantly reduced corneal edema. The vehicle group displayed significantly more corneal thickness and haze, which is indicated by the bright white area in the central cornea (top image, arrow).

Histological analysis confirmed that CPN 0.1% treated eyes had a decrease in incidence and severity of corneal stromal findings (fibroplasia, mononuclear cell infiltration, edema, and neovascularization), and corneal epithelial hyperplasia/hypertrophy. CPN 0.1% rapidly and markedly reduces ocular inflammation, corneal opacity and corneal stromal haze while also increasing the rate of corneal healing. The nanoparticle suspension formulations of clobetasol propionate described herein are useful for long-term improvement of vision and visual acuity following ocular alkali burn injury.

### Example 3. Treatment of Skin Burn Wound

The efficacy of CPN 0.1% to reduce inflammation and granulation tissue formation was evaluated in a pig burn wound model (n=3 female Yorkshire-cross pigs, 35±10 kg). On day 0, animals were sedated with tiletamine-zolazepam followed by isoflurane anesthesia. Ten full thickness 2 cm diameter burn wounds were induced on the back of the animals using a 2 cm diameter brass rod (4.8 cm in height, 100 g) heated to 100° C. To induce the burn wounds, the rod was quickly dried, and placed on the surface of the skin for 45 sec with no additional pressure applied. Wounds were debrided/excised with a 2 cm trephine, dressed with transparent film dressing (Tegaderm™) followed by a blue absorbent pad, and then the torso was wrapped in elastic bandaging. Post-surgical pain was managed with buprenorphine (0.02 mg/kg, IM), and a fentanyl patch (50 μg per hour) was secured to shaved skin on days 0, 4, 7, and 11.

Wounds from one animal served as untreated control wounds, and wounds on two animals were treated with CPN 0.1% (i.e., n=20 wounds CPN 0.1% group; n=10 wounds untreated control group). CPN 0.1% treatment was initiated on day 0 after wound induction, with additional treatments on days 2, 4, 7, and 9. On treatment days, CPN 0.1% was applied to the surface of the wound by injection through the Tegaderm™ dressing with a sterile syringe and needle at a volume of 1.5 mL on days 0-4 and 1 mL on days 7-9. On treatment days, assessments of the wounds were made using a modified Draize scoring system to evaluate erythema and edema. The degree of granulation tissue was assessed using a scoring system with a score of 1 representing no granulation tissue present, 2 indicating granulation tissue partially covering the base of the wound, 3 indicating granulation tissue completely covering the base of the wound but not filling the wound volume, and 4 indicating that granulation tissue is present throughout the volume of the wound.

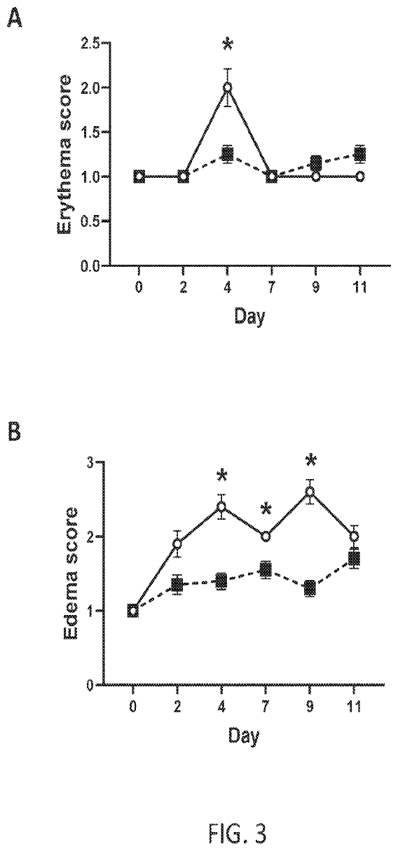

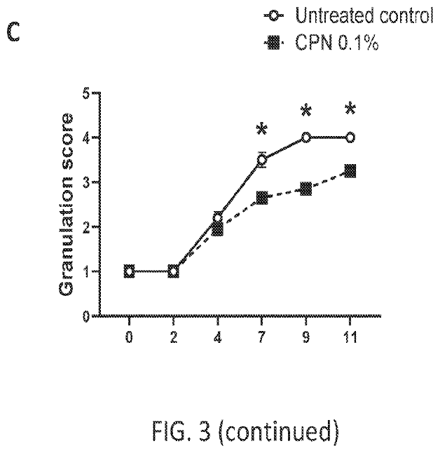

The results are shown in FIG. 3. CPN 0.1% reduced scores of inflammation (erythema and edema) after only 2 treatments, and significantly reduced the amount of granulation tissue on days 7-11 (two-way ANOVA followed by Bonferroni's post hoc test). CPN 0.1% treatment rapidly reduces erythema (Panel A) and edema (Panel B) by day 4, with edema being significantly reduced through day 9, compared to control wounds, indicating accelerated improvement in wound healing. CPN 0.1% also reduced granulation tissue formation on days 7-11 supporting a reduction in hypertrophic scar formation (Panel C). Up to 90 percent of burn wounds result in hypertrophic scarring in humans, and extensive and prolonged inflammation and excessive granulation tissue formation are thought to be critical drivers of hypertrophic scar formation. The nanoparticle suspension formulations of clobetasol propionate described herein are useful for reducing hypertrophic scar formation from burn wounds.

### Example 4. Treatment and Reduction of Hypertrophic Scarring

The efficacy of CPN 0.1% to reduce hypertrophic scarring was evaluated in a rabbit ear hypertrophic scar model (n=2 New Zealand White rabbits, 2.5-3 kg). One rabbit was treated with CPN 0.1% (n=8 wounds), and the other rabbit served as an untreated control (n=8 wounds). On day 0, animals were anesthetized with ketamine/xylazine and four 7 mm diameter full thickness excision wounds were surgically produced on the ventral side of both ears of each rabbit. CPN 0.1% once daily treatment was initiated within 60 minutes of wound induction and continued for 5 days per week for 3 weeks. Treatments consisted of 50 μL CPN 0.1% applied onto the wound, followed by covering with a sterile 8×8 mm piece of filter paper, and application of an additional 50 μL of CPN 0.1% to the filter paper. Wounds were bandaged with a round Band Aid adhesive dressing followed by transparent film dressing (Tegaderm™). Untreated control wounds were dressed exactly the same way, except that no treatment was applied. Two times per week, wounds were assessed for inflammation using a Draize scoring system where an erythema score of 0 indicates no erythema, 1 indicates slight erythema, 2 indicates well defined erythema, 3 indicates moderate to severe erythema and 4 indicates severe erythema or eschar. An edema score of 0 indicates no edema, 1 indicates slight edema, 2 indicates slight edema with well-defined edges, 3 indicates edema (raised >1 mm) and 4 indicates severe edema (raised >1 mm and extending beyond the treatment site). On day 28 (7 days after the last treatment), the animals were euthanized and wounds were collected, fixed and stained with Masson's trichrome for histological evaluation.

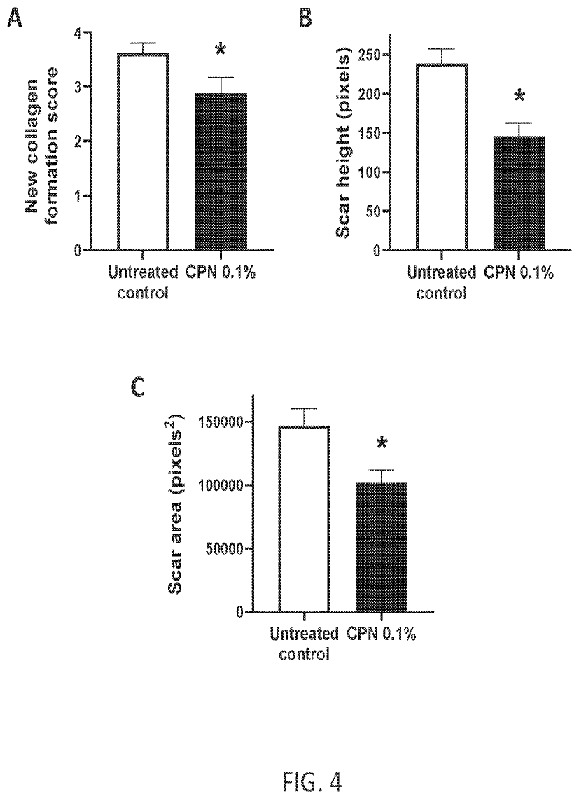

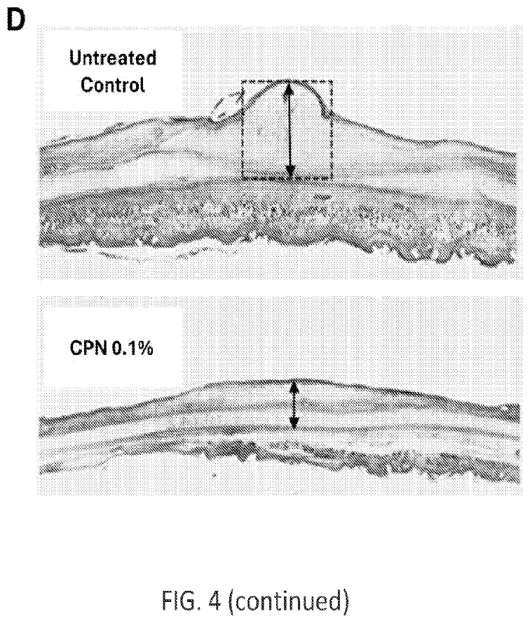

The results are shown in FIG. 4. During the first week of treatment, CPN 0.1% rapidly and significantly reduced erythema [two-way ANOVA, F (8, 112)=3.695, p=0.0007] and edema [two-way ANOVA, F (8, 112)=4.680, p<0.0001] compared to control. On day 28, histological analysis showed that the untreated control group displayed hypertrophic scaring for all wounds, indicated by elevated scar formation. CPN 0.1% treatment significantly reduced new collagen formation (Panel A), scar height (Panel B), and scar area (Panel C) compared to controls (Student's unpaired t-test). Panel D shows representative images on Day 28 of an untreated wound and a wound treated with CPN 0.1%. The overall size of the scar was determined by measuring the pixel area of the scar, and the scar elevation was determined by the pixel height of the scar from the base of the wound to the top of the epidermis. The scar height was measured at the arrows. There is clear evidence of a hypertrophic scar in the untreated control wound (top image, hatched box) and there is an absence of hypertrophic scarring in the wound treated with CPN 0.1% (bottom image). The nanoparticle suspension formulations of clobetasol propionate described herein significantly and substantially reduces and/or prevents abnormal hypertrophic scaring from a wound.

### Example 5. Reduction or Removal of Abnormal Hypertrophic Scarring

The efficacy of intralesional injection of the nanoparticle suspension formulations of clobetasol propionate described herein to reduce hypertrophic scarring is evaluated in a rabbit ear hypertrophic scar model. For example, on day 0, New Zealand White rabbits (n=4 CPN 0.1% group; n=4 vehicle group) are anesthetized with ketamine/xylazine and four 7 mm diameter full thickness excision wounds are surgically produced on the ventral side of both ears. The wounds are untreated for 20 days, by which time, the wounds are closed and develop hypertrophic raised scars. On days 21, 28 and 35 CPN 0.1% or vehicle are injected (10 μL) into the scar using a 32-gauge needle and a microsyringe pump (Hamilton). On day 40, the animals are euthanized and wounds excised, fixed and stained with Masson's trichrome for histological evaluation of collagen and fibrocyte architecture.

Histological analysis is used to compare hypertrophic scaring in the wounds, indicated by elevated scar formation and disorganized collagen and fibrocyte architecture. Intralesional injection of the suspension formulations of clobetasol propionate nanoparticles described herein is expected to significantly reduce new collagen formation, scar height, abnormal fibrocyte architecture and scar area compared to controls, resulting in the absence of hypertrophic scars, showing that intralesional injection of the suspension formulations of clobetasol propionate nanoparticles described herein can significantly reduce the size of abnormal hypertrophic scars from a wound.

### Example 6. Treatment of Tenosynovitis, Tendinitis, Enthesitis and Arthritis

The efficacy of CPN 0.1% to reduce arthritis symptoms was evaluated in a model of collagen-induced arthritis (CIA) in rats. To induce the arthritic phenotype, male Lewis rats (n=20, 200-250 g) were administered a subcutaneous injection of 200 μL of 1 mg/ml bovine type II collagen/Incomplete Freund's adjuvant solution at the base of the tail on day 0, followed by a booster injection of 100 μL on day 7. The CIA model produces an arthritic phenotype in one or both hindlimbs of rats by day 28-42, resulting in swollen paws with inflamed joints and tendons. Once the arthritic phenotype was developed, CPN 0.1% or vehicle was injected (50 μL) subcutaneously into the pad of both hind paws once daily for 14 days (randomized as n=10 CPN 0.1% group; n=10 vehicle group). Hind paws were assessed daily for inflammation using a clinical score of 0-4, with 0 being normal, 1 indicating mild redness and swelling of the ankle or digits, 2 indicating moderate redness and swelling of the ankle and paw, 3 indicating severe redness and swelling of the entire ankle and paw including digits and 4 indicating a maximally inflamed hind limb with involvement of multiple joints. Plethysmometry was used to measure paw volumes twice a week. On day 15 rats were assessed for paw and joint inflammation followed by euthanasia.

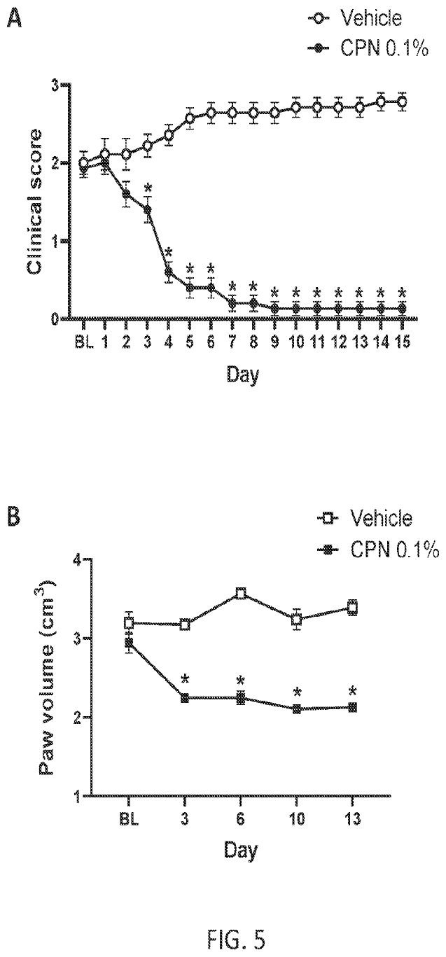

The results are shown in FIG. 5. CPN 0.1% rapidly and significantly reduced clinical scores of hind paw inflammation (Panel A) and hind paw volume (Panel B) by day 3 of treatment compared to vehicle (two-way ANOVA followed by Bonferroni's post hoc test). These effects were maintained throughout the observation period. The paws of rats treated with CPN 0.1% appeared normal within 7 days of treatment, while vehicle-treated animals had reduced mobility due to paw, joint and tendon inflammation for the duration of the observation period. CPN 0.1% rapidly and significantly reduced paw swelling and joint and tendon inflammation, indicating that the suspension formulations of clobetasol propionate nanoparticles described herein are useful in reducing inflammation around the joints, tendons and ligaments in subjects with arthritis, tenosynovitis, tendinitis and enthesitis.

### Example 7. Treatment of Inner Ear Disorder (Hearing Loss)

The efficacy of the suspension formulations of clobetasol propionate nanoparticles described herein is assessed in a noise-induced hearing loss model. For example, C57BL/6J mice (n=10 CPN 0.1% group; n=10 vehicle group) are housed in a noise-controlled environment throughout the study. Baseline hearing thresholds are measured with bilateral auditory brainstem responses (ABR) and distortion product otoacoustic emissions (DPOAEs) at 5-40 kHz. On day 0, animals are anesthetized with isoflurane and receive an intratympanic (IT) injection of 5 μL of CPN 0.1% or the vehicle into the ears. IT injections are performed with the animal secured in a stereotaxic apparatus equipped with a microsyringe/microinjection pump assembly. Following IT injection, the animals are held in position with the injected ear facing up for 30 min to allow the dosing suspensions to contact the round window membrane and clobetasol propionate to diffuse into the inner ear fluid.

At 1.5 h after IT injection, the animals are subjected to a single noise exposure in a sound-proof room. Noise exposure is performed with the mouse in a wire cage surrounded by ceramic speakers and noise is generated and filtered to 4.0-45.0 kHz. Mice are exposed one time to 110 dB for 30 min. Two weeks after noise exposure, ABR assessments of hearing thresholds are performed, followed by euthanasia and collection of cochleae for histological analysis of the auditory apparatus of the inner ear.

Histological analysis of cochleae is used to compare the level of preservation of inner and outer hair cell counts and their neural connections in the treatment and vehicle-treated groups. IT administration of the formulations described herein is expected to show significantly less ABR threshold shifts, indicating that the formulations prevent noise-induced hearing loss compared to vehicle injection showing that the suspension formulations of clobetasol propionate nanoparticles are useful for treating noise-induced hearing loss by reducing cochlear hair cell loss and preserving hearing.

### Example 8. Treatment of Inner Ear Disorder (Tinnitus)

The efficacy of IT injection of the suspension formulations of clobetasol propionate nanoparticles described herein to reduce tinnitus is evaluated in a mouse model. For example, C57BL/6J mice (n=10 CPN 0.1% group; n=10 vehicle group) are tested using a sound-based avoidance detection (SBAD) paradigm. Mice are trained in a shuttle box to perform a Go or No-Go operant response using foot shock (0.1-0.4 mA) as negative reinforcement. The shuttle box has two chambers of the same size with a shutter door in the middle and light beams throughout the shuttle box to detect the location of the animal. The Go trial monitors motor, motivation, and learning/memory functions, and the No-Go trial monitors tinnitus-like behavior. Both types of trials are randomly assigned throughout each training and testing session with a 4 second inter-trial interval. Training sessions start with a 2 minute acclimation period followed by 100 trials per day for a total of 30-40 minutes. In Go trials, upon presentation of any sound stimulus, animals are trained to move from one compartment to the other. When they move to the other compartment, the shutter is closed. If they do not move through the shutter, they receive a foot shock 5 seconds after the start of the sound stimulus. In No-Go trials, animals are trained to stay in the compartment if no sound is delivered. If they go through the shutter within 10 seconds, they receive a shock but the shutter remains open. The shock is turned off after the animal moves to the other chamber or at the end of the trial for both Go and No-Go trails. Sound stimuli are randomly presented and vary in both frequency and intensity.

During testing, foot shock continues to be applied after errors in Go trials, and the shutter is closed as soon as the animal moves to the other compartment. However, in No-Go trials, a shock is no longer administered and the shutter is closed right away if an animal crosses into the other compartment. Thus, in No-Go trials, moving from one compartment to the other compartment in the absence of an external sound stimulus indicates that the animal has tinnitus.

After successfully completing SBAD training (90-100% correct trial responses), mice are subjected to unilateral noise-induce trauma to produce tinnitus. On Day 0 before noise exposure, mice are anesthetized with isoflurane and receive an IT injection (5 L) of CPN 0.1% or vehicle into the right ear. IT injections are performed with the animal secured in a stereotaxic apparatus equipped with a microsyringe/microinjection pump assembly. Following IT injection, the animals are held in position with the injected ear facing up for 30 minutes to allow the dosing suspensions to contact the round window membrane and allow diffusion of clobetasol propionate into the inner ear fluid. The left ear is blocked with a modified ear plug (Decidamp2™), held in place with Stomahesive™ (ConvaTec). Two hours later, mice are placed in a sound-attenuating chamber and exposed to 120 dB broadband noise (4-25 kHz) for 2 h.

To assess the presence of tinnitus, mice undergo SBAD testing at 2 weeks, 1 month, and 2 months after noise exposure. At each timepoint the vehicle and treatment groups display little error in the Go trials, indicating normal motivation, motor, and memory/learning function. However, in No-Go trials, the vehicle group displays a larger percentage of errors, i.e. the mice move from one compartment to the other in the absence of an external sound stimulus, compared to the treatment group, indicating the presence of tinnitus. Efficacy is demonstrated when correct responses in No-Go trials, i.e. not moving from one compartment to the other in the absence of an external sound stimulus, are 20% or higher. Displaying few errors (90-100% correct responses) in No-Go trials indicates a high degree of efficacy in reducing noise-induced tinnitus. IT injection of the suspension formulations of clobetasol propionate nanoparticles described herein is expected to show a high percentage of correct responses showing that the formulations are useful in treating tinnitus in subjects, including humans.

### Example 9. Treatment of Allergic Rhinitis/Sinusitis

The suspension formulations of clobetasol propionate nanoparticles described herein are useful for treating subjects with allergic rhinitis. For example, a 30 year old female visits a clinic complaining of profuse nasal drip, nasal itch and inability to breathe through the nose that have not improved with conventional allergy therapies. The clinician observes the following signs/symptoms which are characteristic of allergic rhinitis: nasal exudate, deep redness of the nasal mucosa and marked swelling of the nasal and sinus mucous membranes. The clinician recognizes the problems with conventional allergy therapies that may not be effective in treating the subject's symptoms and signs, particularly when there is marked exudation of fluid. In this example, the clinician applies an aqueous suspension of nanoparticles of clobetasol propionate to the nasal and sinus mucosa using a spray bottle. The liquid suspension is applied by spraying as evenly as practical to the entire mucosal surface of the nose and allowed to settle for five to ten minutes. Thereafter, the liquid suspension is re-applied once or more frequently daily over a period of one week or more. After one week or longer, the subject returns to the clinic for evaluation. The subject acknowledges marked improvement in the symptoms and signs of allergic rhinitis and remarks to the clinician that the treatment reduced the itch and difficulty in breathing through the nose. Further observations by the clinician confirm faster resolution of the rhinitis with the suspension of nanoparticles of clobetasol propionate. The suspension of clobetasol propionate nanoparticles is demonstrated to be effective, convenient and comforting for treating allergic rhinitis.

### Example 10. Treatment of Asthma

The suspension formulations of clobetasol propionate nanoparticles described herein are useful for treating subjects with asthma. For example, a 15 year old female visits a clinic complaining of shortness of breath, chest tightness and pain, wheezing and coughing. The clinician recognizes these symptoms as being consistent with asthma, and applied conventional treatments to rapidly reduce acute asthma symptoms. In this example, the clinician shows the subject how to administer an aqueous suspension of nanoparticles of clobetasol propionate described herein for the treatment of asthma using an inhaler. The liquid suspension is inhaled into the lungs using an inhaler once or more times daily to treat the asthma symptoms. After one week or more, the subject returns to the clinic for evaluation. The subject acknowledges marked improvement in the symptoms of asthma and is currently breathing freely, without asthma symptoms. Further observations by the clinician confirm effective treatment of asthma with clobetasol propionate. The suspension of clobetasol propionate nanoparticles is demonstrated to be effective, convenient and comforting for treating asthma.

### Example 11. Tissue Penetration, Effect of Methyl Cellulose Concentration

Tissue penetration of suspension formulations of clobetasol propionate nanoparticles into conjunctiva was measured using a conventional protocol.

TABLE 3
Concentrations of clobetasol propionate measured in ocular tissues
after one drop (50 μL) of suspension formulation of clobetasol
propionate nanoparticles instilled into the eye of rabbits.

| | Clobetasol Propionate in Conjunctiva | |
|---|---|---|
| Formulation | Cₘₐₓ (ng/g) | Half Life (hr) |
| 0.05% Clobetasol Propionate | 1720 | 4.52 |
| with 0.5% Methyl Cellulose, as | | |
| described herein (CPN 0.05%, | | |
| Table 2) | | |
| 0.05% Clobetasol Propionate | 970 | 1.34 |
| with 0.2% Methyl Cellulose | | |
| (Comparative Example*) | | |

Cₘₐₓ (ng/g) = Peak concentration (ng per gram of tissue)
*The composition includes the same level of all other excipients listed in Table 2 for CPN 0.05%, except for methyl cellulose.

Suspensions of clobetasol propionate nanoparticles described herein provide a longer half-life (i.e., retention of the active ingredient, clobetasol propionate, in the target tissue for pharmacological activity, such as conjunctiva, after topical ocular surface administration. In addition, the suspensions of clobetasol propionate nanoparticles described herein provide higher concentration (Cₘₐₓ) and therapeutic exposure of clobetasol propionate in the target tissue at equivalent dose of the active ingredient in the formulations.

### Example 11. Tissue Penetration, Effect of Boric Acid and EDTA

Tissue penetration of suspension formulations of clobetasol propionate nanoparticles into cornea was measured using a conventional protocol. Concentrations of clobetasol propionate measured in target tissue for pharmacological activity after formulation described herein (Table 4) containing boric acid and edetate disodium dehydrate (EDTA) after topical ocular surface administration of one drop (50 μL) of 0.05% clobetasol propionate nanoparticle formulation shown in Table 2.

TABLE 4
Concentrations of clobetasol propionate measured in cornea after
one drop (50 μL) of suspension formulation of clobetasol
propionate nanoparticles instilled into the eye of rabbits

| | Clobetasol Propionate | |
|---|---|---|
| | in Cornea | |
| | Dose Normalized | Half |
| Formulation | Cₘₐₓ (ng/g) | Life (hr) |
| 0.05% Clobetasol Propionate with Boric | 3240 | 5.42 |
| Acid and EDTA, as described herein | | |
| (CPN 0.05%, Table 2) | | |
| 0.1% Clobetasol Propionate free of Boric | 2100 | 5.34 |
| Acid and EDTA (Comparative Example)* | | |

Cₘₐₓ (ng/g) = Peak concentration (ng per gram of tissue)
*The composition includes the same level of all other excipients listed in Table 2 for CPN 0.1%, except being free of boric acid and EDTA.

Suspensions of clobetasol propionate nanoparticles containing boric acid and EDTA described herein provide a higher concentration and therapeutic exposure of clobetasol propionate in the targeted tissue, such as cornea, at equivalent dose of the active ingredient. The different dose amounts, 0.05% and 0.1%, were normalized when presenting the Cₘₐₓ value.

All patents, patent publications, and other publications referenced and identified in the present specification are individually and expressly incorporated herein by reference in their entirety for the purpose of further describing the invention. However, the cited patents, patent publications, and other publications should not be construed as admitted prior art.

1.-30. (canceled)

31. A method for treating inflammation associated with a trauma, injury or wound, inner ear disorder, or respiratory disorder in a subject, the method comprising administering a composition comprising a sterile suspension formulation of nanoparticles of clobetasol propionate to the subject.

32. The method of claim 31 for treating a joint injury, dermal injury, or ocular burn.

33. The method of claim 32 wherein the joint injury is tenosynovitis, tendinitis, enthesitis, or arthritis.

34. The method of claim 31 wherein the suspension formulation is capable of reducing the complications of tenosynovitis, tendinitis, enthesitis or arthritis, including pain, discomfort, or decreased mobility, or improving joint, tendon, or ligament function.

35. The method of claim 31 for treating abnormal wound healing, hypertrophic scarring, keloid formation, corneal scarring, or impaired vision.

36. The method of claim 31 wherein the suspension formulation is capable of reducing the complications of a dermal injury, ocular burn, or abnormal wound healing.

37. The method of claim 31 wherein the complications include inflammation, pain, abnormal granulation, scarring, keloid formation, contracture, deformity, pigmentation, or impaired vision.

38. The method of claim 31 wherein the inner ear disorder is hearing loss, tinnitus, or vertigo.

39. The method of claim 31 wherein the respiratory disorder is allergic rhinitis, sinusitis, or asthma.

40. The method of claim 31 wherein the suspension formulation is configured for application to the trauma, injury, or wound using a spray bottle, dropper bottle or aerosol device.

41. The method of claim 31 wherein the sterile suspension formulation is configured for injection into the trauma, injury, or wound with a syringe and needle.

42. The method of claim 31 wherein the suspension formulation is capable of reducing inflammation or inner ear dysfunction.

43. The method of claim 31 wherein the suspension formulation is configured for administration to the skin, eye, nose, respiratory tract, nasal mucosa, tympanic membrane, middle ear, tympanic cavity, synovial space, ligament, or joint once or more times daily for up to 52 weeks.

44. A drop, spray, mist, aerosol, gel, or injection formulation comprising a suspension of nanoparticles of clobetasol propionate.

45. The drop, mist, spray, aerosol, gel, or injection formulation of claim 44 further comprising sodium chloride, hydrogenated soybean, lecithin, anhydrous citric acid, glycerin, Poloxamer 407, polyvinyl alcohol (PVA), boric acid, edetate disodium dihydrate, benzalkonium chloride, methylcellulose, or trisodium citrate, or a combination thereof.

46. The drop, mist, spray, aerosol, gel, or injection formulation of claim 44 further comprising an antioxidant, an antimicrobial agent, an anti-inflammatory, an anesthetic, an analgesic, or a lubricant, or a combination thereof.

47. The injection formulation according to claim 44, wherein the suspension of nanoparticles is configured for injection into a hypertrophic scar or keloid, injection into the middle ear or a tympanic cavity, or injection into a joint, synovial space, tendon, or ligament of the subject.

48. A unit dose comprising a sterile suspension of nanoparticles of clobetasol propionate, where the unit dose is configured for administration by injection, topical dermal application, intra-articular injection, intradermal injection, inhalation, instillation in the nasal cavity, intratympanic injection, or ocular instillation.

49. The unit dose of claim 48 further comprising sodium chloride, hydrogenated soybean, lecithin, anhydrous citric acid, glycerin, Poloxamer 407, polyvinyl alcohol (PVA), boric acid, edetate disodium dihydrate, benzalkonium chloride, methylcellulose, or trisodium citrate, or a combination thereof.

50. The unit dose of claim 48 further comprising an antioxidant, an antimicrobial agent, an anti-inflammatory, an anesthetic, an analgesic, or a lubricant, or a combination thereof.
